(19) United States
(12) Patent Application Publication
(10) Pub. No.: US 2019/0240435 A1
(43) Pub. Date: Aug. 8, 2019

(54) BREATHING ASSIST SYSTEM INCLUDING AN ORAL APPLIANCE AND CONNECTOR SYSTEM THEREFOR

(71) Applicant: Oventus Medical Limited, Indooroopilly (AU)

(72) Inventors: Neil ANDERSON, Roseville, New South Wales (AU); Christopher Patrick HART, Indooroopilly (AU); Anthony IGNACIO, Bossley Park, New South Wales (AU); Zoran MILIJASEVIC, Bayview, New South Wales (AU)

(21) Appl. No.: 16/340,519

(22) PCT Filed: Oct. 10, 2017
(86) PCT No.: PCT/AU2017/051092

§ 371 Date: Apr. 9, 2019

(30) Foreign Application Priority Data
Oct. 10, 2016 (AU) 2016904167

(51) Int. Cl. A61M 16/06; A61M 16/20; A61M 16/00; A61M 16/08

(52) U.S. Cl. CPC A61M 16/06; A61M 16/208; A61M 16/0666; A61M 16/024; A61M 16/0003; A61M 16/0875; A61M 2230/63; A61M 2230/62; A61M 2016/0027; A61M 2016/0033; A61M 2205/3368; A61M 2205/36; A61M 2016/0661; A61M 2205/8206

(57) ABSTRACT

A system for providing breathing assistance to a user includes (a) an oral appliance including a body for positioning within an oral cavity of the user, the body defining at least one extra-oral opening for allowing airflow between lips of the user in fluid communication with at least one intra-oral opening provided in the oral cavity to direct airflow into and/or out of a posterior region of the oral cavity; and, (b) at least one of: (i) a housing connected to the at least one extra-oral opening of the appliance, the housing configured to allow natural breathing through the appliance; and, (ii) a connector system for receiving air from a positive airway pressure (PAP) device, the connector system connected to the at least one extra-oral opening of the appliance.

### PRIORITY DOCUMENTS

The present application claims priority from Australian Provisional Application No. 2016904167 titled “A BREATHING ASSIST SYSTEM INCLUDING AN ORAL APPLIANCE AND CONNECTOR SYSTEM THEREFOR” filed on 10 Oct. 2016, the content of which is hereby incorporated by reference in its entirety.

### BACKGROUND OF THE INVENTION

The present invention relates to a breathing assist system, and in one example to a system including an oral appliance and connector system connected to a positive airway pressure (PAP) device.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Poor quality or ineffective breathing is an issue which can affect the performance of people in their day to day activities either while they are awake and/or when they are asleep. While awake this can be less optimal performance in activities such as sport or even while performing everyday tasks. While asleep breathing disorders can lead to snoring and/or sleep apnoea.

Snoring arises due to vibration of soft tissues within the respiratory pathways of an individual, and is typically caused by obstructed air movement during breathing while sleeping. Snoring can arise from a range of different physical causes such as blocked sinuses, and typically occurs when the muscles of the upper throat relax during sleep.

Snoring can also be associated with Obstructive Sleep Apnoea (OSA), which is caused by obstruction of the upper airway and results in repetitive pauses in breathing during normal sleep. Individuals having OSA often suffer from daytime sleepiness and fatigue associated with significant levels of sleep disturbance, whilst a partners sleep patterns are also often disturbed by associated snoring.

Current therapy for treatment of OSA can include lifestyle changes, the use of mechanical devices, such as oral or nasal devices that augment the airway, surgical procedures to enlarge and stabilize the airway during sleep, and continuous or variable positive airway pressure (PAP) devices.

However, surgical procedures can be severe and are not therefore widely used unless absolutely necessary. Whilst PAP devices have had a positive impact, these can be uncomfortable to wear for prolonged time periods, are expensive, and are often noisy, which can in turn lead to additional sleep disturbance. As a result, surgery and PAP treatment have had limited application in treating sleep apnoea, and are not generally considered appropriate treatment for snoring.

It has been shown that approximately 30-50% of continuous variable positive airway (CPAP) device users are non-compliant users with 2 years of starting their treatment. CPAP systems deliver airflow to a mask which the user typically wears over their mouth and nose. CPAP masks suffer from several drawbacks including leakage and discomfort and often users experience a degree of claustrophobia whilst wearing the mask.

Furthermore, as CPAP systems must supply air at sufficient pressure to maintain an airway and act as a pneumatic splint, relatively high pressures are typically required. In addition, high flow rates are required as the mask supplies all of the air for a user during inhalation. In order to achieve such high pressures and flow, relatively large and noisy pumps such as air blowers are conventionally used.

It would therefore be desirable to provide a system in which CPAP pressure and/or air flow rates can be minimised in order to reduce noise, vibration and size of pump and in order to improve comfort and portability.

### SUMMARY OF THE PRESENT INVENTION

In a first broad form, an aspect of the present invention seeks to provide a system for providing breathing assistance to a user, the system including:
a) an oral appliance including a body for positioning within an oral cavity of the user, the body defining at least one extra-oral opening for allowing airflow between lips of the user in fluid communication with at least one intra-oral opening provided in the oral cavity to direct airflow into and/or out of a posterior region of the oral cavity; and,
b) at least one of:
   i) a housing connected to the at least one extra-oral opening of the appliance, the housing configured to allow natural breathing through the appliance; and,
   ii) a connector system for receiving air from a positive airway pressure (PAP) device, the connector system connected to the at least one extra-oral opening of the appliance.

In one embodiment, the at least one extra-oral opening is provided as part of at least one connection portion that at least partially protrudes from the appliance.

In one embodiment, the at least one connection portion is tubular.

In one embodiment, the connector system includes a connector having a body that defines at least one outlet that connects to the at least one connection portion of the oral appliance.

In one embodiment, the body further defines at least two channels, each channel connecting an intra-oral opening to the at least one extra-oral opening, each channel passing at least one of at least partially along the buccal cavity and at least partially between the teeth to thereby provide an airway for the user, the airway at least partially bypassing the nasal passage and acting to replicate a healthy nasal passage and pharyngeal space.

In one embodiment, air from the PAP is delivered to the user through at least one of:
a) the nose of the user;
b) the oral appliance via the airway; and,
c) the nose and the oral appliance via the airway.

In one embodiment, at least one of the extra-oral openings defines a breathing port that is operable to allow natural breathing through the appliance.

In one embodiment, the connector body includes a vent in fluid communication with the breathing port.

In one embodiment, at least one of the breathing port and vent in the connector body includes at least one of an adjustable or one-way valve for controlling natural breathing.

In one embodiment, the channel on each side of the appliance is partitioned so as to define a first and second airway.

In one embodiment, the body of the appliance defines:
a) a first extra-oral opening in fluid communication with a first intra-oral opening via the first airway; and,
b) a second extra-oral opening in fluid communication with a second intra-oral opening via the second airway.

In one embodiment, the connector body is connected to the first extra-oral opening so that air from the PAP device is directed into the appliance through the first airway.

In one embodiment, the second airway is used for at least one of:
a) natural breathing; and,
b) exhalation.

In one embodiment, the first airway extends along the user's buccal cavity and the second airway extends at least one of:
a) between the user's maxillary and mandibular teeth; and,
b) between the user's maxillary and mandibular teeth and along the user's buccal cavity.

In one embodiment, air pressure and/or flow can be delivered separately and/or in conjunction to the respective first and second airways.

In one embodiment, the connector system includes a connector body defining at least one passageway between an inlet for receiving air from the PAP device and one or more outlets for directing air into the appliance and/or nose of the user.

In one embodiment, at least one inlet valve is provided downstream of the inlet for regulating pressure and/or flow through the connector.

In one embodiment, a first inlet valve is provided to regulate pressure and/or flow into the appliance and a second inlet valve is provided downstream of the first inlet valve for regulating pressure and/or flow into a nasal connecting portion that directs air into the nose of the user.

In one embodiment, the connector body includes one or more first outlets that connect to one or more corresponding first extra-oral openings of the appliance to allow air from the PAP device into the appliance.

In one embodiment, the connector body includes one or more openings that connect to one or more corresponding second extra-oral openings of the appliance to facilitate natural breathing.

In one embodiment, the connector body includes one or more breathing ports in fluid communication with the one or more openings.

In one embodiment, the nasal connecting portion is connected to one or more second outlets of the connector body.

In one embodiment, nasal prongs are connected to the nasal connection portion.

In one embodiment, the nasal connecting portion and/or nasal prongs are at least one of rotatably and/or slidably movable relative to the connector body.

In one embodiment, nasal pillows are inserted into the nasal prongs.

In one embodiment, the nasal pillows are slidably movable with respect to the nasal prongs.

In one embodiment, exhalation ports are provided in the nasal connecting portion and/or nasal prongs.

In one embodiment, the system further includes a PAP device.

In one embodiment, the PAP device is battery operated.

In one embodiment, the PAP device includes a diaphragm air pump.

In one embodiment, the PAP device is mounted on:
a) the connector system in front of the mouth;
b) the oral appliance;
c) the arm, head, neck or chest of the user.

In one embodiment, one or more sensors are provided in or on the connector body and/or nasal connecting portion for measuring an indication of at least one of:
a) body or head position;
b) air pressure;
c) air flow rate;
d) temperature;
e) moisture; and,
f) motion.

In one embodiment, the one or more sensors are positioned proximate the one or more breathing and/or exhalation ports in the connector body and/or nasal connecting portion.

In one embodiment, the housing connected to the at least one extra-oral opening that permits natural breathing includes at least one of:
a) a valve/restrictor; and,
b) a heat and moisture exchanger.

In one embodiment, the housing includes one or more sensors for measuring an indication of at least one of:
a) body or head position;
b) air pressure;
c) air flow rate;
d) temperature;
e) moisture; and,
f) motion.

In one embodiment, the system further includes at least one electronic processing device coupled to the one or more sensors for:
a) determining sensor data indicative of signals from each of the one or more sensors; and,
b) causing the sensor data to be wirelessly transmitted to a monitoring device.

In one embodiment, the sensor data is stored in a remote data store.

In one embodiment, the at least one electronic processing device is configured to control at least one of the PAP device and one or more valves in the flow path in accordance with the determined sensor data to thereby regulate pressure and/or flow provided to the user.

In one embodiment, the connector system includes:
a) an interface that is coupled to an extra-oral portion of the appliance that extends beyond the lips of the user in which the at least one extra-oral opening is provided;
b) an adaptor which is coupled to the interface;
c) a nasal tube holder which is coupled to the adaptor; and,
d) a nasal tube for receiving air from the PAP device that is removably attached to the nasal tube holder and which allows PAP to be delivered to the nose of the user.

In one embodiment, the nasal tube holder includes one or more breathing ports in a front surface thereof in fluid communication with the at least one extra-oral opening of the appliance.

In one embodiment, a one-way valve and/or heat and moisture exchanger is positioned behind one of the breathing ports of the nasal tube holder.

In a second broad form, an aspect of the present invention seeks to provide an apparatus for providing breathing assistance, the apparatus including a body for positioning within an oral cavity of a user, the body defining:
a) at least one first and second extra-oral opening configured to allow airflow between lips of the user;
b) at least two intra-oral openings provided in the oral cavity to allow air flow into and out of a posterior region of the oral cavity; and,
c) at least two channels, each channel connecting a respective intra-oral opening to at least one of the first and second extra-oral openings and each channel passing at least one of at least partially along the buccal cavity and at least partially between the teeth to thereby provide an airway for the user, the airway at least partially bypassing the nasal passage and acting to replicate a healthy nasal passage and pharyngeal space.

In one embodiment, a first airway is provided between a first extra-oral opening and a first intra-oral opening and a second airway is provided between a second extra-oral opening and a second intra-oral opening, and wherein the first airway is partitioned from the second airway.

In one embodiment, the first airway passes at least partially along the buccal cavity and the second airway passes at least one of:
a) at least partially between the user's maxillary and mandibular teeth; and,
b) at least partially between the user's maxillary and mandibular teeth and at least partially along the buccal cavity.

In one embodiment, the first airway and second airway are partitioned so that the first airway is positioned at least one of:
a) above the second airway; and,
b) to the outer lateral side of the second airway.

In one embodiment, the first airway is used during inhalation and the second airway is used during exhalation.

In one embodiment, the at least one first extra-oral opening is connectable to a supply of air from a positive airway pressure (PAP) device.

In one embodiment, the at least one first extra-oral opening is connectable to a connector system including a connector having a body in fluid communication with an inlet for receiving air from the PAP device and at least one outlet connected to the at least one first extra-oral opening for supplying the air thereto.

In one embodiment, the at least one second extra-oral opening is connectable to at least one opening in the connector body thereby allowing natural breathing through at least part of the connector.

In one embodiment, the at least one second extra-oral opening is connectable to a sensor housing having at least one sensor for measuring an indication of at least one of:
a) body or head position;
b) air pressure;
c) air flow rate;
d) temperature;
e) moisture; and,
f) motion.

In one embodiment, the at least one first and second extra-oral openings are provided in an extra-oral portion of the apparatus that extends beyond the lips of the user and has a generally elliptic cross section.

In a third broad form, an aspect of the present invention seeks to provide a connector system for connection to an oral appliance for providing breathing assistance to a user, the connector system including a connector having a body connectable to at least one extra-oral opening of the oral appliance, the body defining at least one passageway between an inlet for receiving air from a positive airway pressure (PAP) device and at least one outlet in fluid communication with the inlet for directing the air from the PAP device into at least one of the at least one extra-oral opening of the oral appliance and the nose of the user.

In one embodiment, at least one inlet valve is provided downstream of the inlet for regulating pressure and/or flow through the connector.

In one embodiment, a first inlet valve is provided to regulate pressure and/or flow into the appliance and a second inlet valve is provided downstream of the first inlet valve for regulating pressure and/or flow into a nasal connecting portion that directs air into the nose of the user.

In one embodiment, the connector body includes one or more first outlets that connect to one or more corresponding first extra-oral openings of the appliance to allow air from the PAP device into the appliance.

In one embodiment, the connector body includes one or more openings that connect to one or more corresponding second extra-oral openings of the appliance to facilitate natural breathing.

In one embodiment, the connector body includes one or more breathing ports in fluid communication with the one or more openings.

In one embodiment, the nasal connecting portion is connected to one or more second outlets of the connector body.

In one embodiment, nasal prongs are connected to the nasal connection portion.

In one embodiment, the nasal connecting portion and/or nasal prongs are at least one of rotatably and/or slidably movable relative to the connector body.

In one embodiment, nasal pillows are inserted into the nasal prongs.

In one embodiment, the nasal pillows are slidably movable with respect to the nasal prongs.

In one embodiment, exhalation ports are provided in the nasal connecting portion and/or nasal prongs.

In one embodiment, the connector system further includes one or more sensors positioned to monitor at least one of pressure and/or air flow to or from the appliance and/or nose.

In one embodiment, the connector body is 3D printed or moulded.

In one embodiment, the connector has a multi-part body including:
a) an interface that is coupled to an extra-oral portion of the appliance that extends beyond the lips of the user in which the at least one extra-oral opening is provided;
b) an adaptor which is coupled to the interface;
c) a nasal tube holder which is coupled to the adaptor; and,
d) a nasal tube for receiving air from the PAP device that is removably attached to the nasal tube holder and which allows PAP to be delivered to the nose of the user.

In a fourth broad form, an aspect of the present invention seeks to provide a system for providing breathing assistance to a user, the system including:
a) an oral appliance including a body for positioning within an oral cavity of the user; and,
b) at least one extra-oral connector coupled to the oral appliance that at least one of:
   i) modifies an airway;
   ii) provides an airway; and,
   iii) monitors an airway.

In one embodiment, the at least one extra-oral connector houses at least one sensor for measuring an indication of at least one of:
a) body or head position;
b) air pressure;
c) air flow rate;
d) temperature;
e) moisture; and,
f) motion.

In one embodiment, the at least one extra-oral connector includes at least one of:
a) a valve/restrictor to provide resistance during exhalation; and,
b) a heat and moisture exchanger.

In one embodiment, the at least one sensor and/or valve is positioned proximate the user's mouth and/or nose.

In one embodiment, the body of the oral appliance defines at least one extra-oral opening for allowing airflow between lips of the user in fluid communication with at least one intra-oral opening provided in the oral cavity to direct airflow into and/or out of a posterior region of the oral cavity.

In one embodiment, the body further defines at least two channels, each channel connecting an intra-oral opening to the at least one extra-oral opening, each channel passing at least one of at least partially along the buccal cavity and at least partially between the teeth to thereby provide an airway for the user, the airway at least partially bypassing the nasal passage and acting to replicate a healthy nasal passage and pharyngeal space.

In one embodiment, the at least one extra-oral connector includes a nasal connecting portion.

In one embodiment, a pair of nasal pillows are coupled to the nasal connecting portion for insertion into a user's nostrils.

In one embodiment, the nasal pillows are custom fit to the user as a result of one of:
a) heat setting a thermoplastic material;
b) bending a flexible tubing.

In one embodiment, the flexible tubing is reinforced by ductile metal strips or coil.

In one embodiment, the at least one extra-oral connector is connected to the at least one extra-oral opening of the appliance and configured to receive air and/or oxygen from a positive airway pressure (PAP) device.

In one embodiment, air and/or oxygen from the PAP device is delivered to the user through at least one of:
a) the nose of the user;
b) the oral appliance via the airway; and,
c) the nose and the oral appliance via the airway.

In one embodiment, the at least one extra-oral connector includes at least one control valve for directing air and/or oxygen to at least one of the mouth and/or nose of the user.

In one embodiment, the at least one extra-oral connector includes at least one vent for controlling natural breathing through the mouth and/or nose.

In one embodiment, the channel on each side of the oral appliance is partitioned so as to define a split airway.

In one embodiment, a first portion of the split airway is used for delivering PAP and a second portion of the split airway is used for natural breathing.

In one embodiment, the extra-oral connector includes:
a) an interface that is coupled to an extra-oral portion of the appliance that extends beyond the lips of the user in which the at least one extra-oral opening is provided;
b) an adaptor which is coupled to the interface;
c) a nasal tube holder which is coupled to the adaptor; and,
d) a nasal tube for receiving air from the PAP device that is removably attached to the nasal tube holder and which allows PAP to be delivered to the nose of the user.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

Examples of the present invention will now be described with reference to the accompanying drawings, in which: —

FIG. 1A is a schematic perspective top side view of a first example of a system for providing breathing assistance;

FIG. 1B is a schematic perspective rear side view of a first example of a connector system used in the system of FIG. 1A;

FIG. 1C is a schematic perspective top side view of a first example an oral appliance used in the system of FIG. 1A;

FIG. 1D is a schematic back view of the oral appliance of FIG. 1C;

FIG. 1E is a schematic sectional view taken through section A-A of FIG. 1D;

FIG. 1F is a schematic front view of the oral appliance of FIG. 1C;

FIG. 1G is a schematic perspective sectional view taken through section B-B of FIG. 1F;

FIG. 1H is a schematic perspective sectional view taken through section C-C of FIG. 1F;

FIG. 2A is a schematic perspective top side view of a second example of a system for providing breathing assistance having a second example of a connector system;

FIG. 2B is a schematic rear view of the system of FIG. 2A;

FIG. 2C is a schematic side view of the system of FIG. 2A;

FIG. 3A is a schematic perspective top side view of a third example of a system for providing breathing assistance having a third example of a connector system;

FIG. 3B is a schematic front view of the system of FIG. 3A;

FIG. 3C is a schematic side view of the system of FIG. 3A;

FIG. 4A is a schematic perspective top side view of a fourth example of a system for providing breathing assistance having a fourth example of a connector system;

FIG. 4B is a schematic side view of the system of FIG. 4A;

FIG. 4C is a schematic front view of the system of FIG. 4A;

FIG. 4D is a schematic perspective view of a second example of an oral appliance used in the system of FIG. 4A showing a housing connected to an extra-oral opening of the appliance;

FIG. 4E is a schematic front view of the oral appliance of FIG. 4D with the housing removed;

FIG. 4F is a schematic sectional view taken through section D-D of FIG. 4E;

FIG. 5A is a schematic perspective top side view of a fifth example of a system for providing breathing assistance having a fifth example of a connector system;

FIG. 5B is a schematic rear view of the system of FIG. 5A showing an airway configuration of a third example of an oral appliance;

FIG. 5C is a schematic sectional view taken through section E-E of FIG. 5B;

FIG. 5D is a schematic rear view of the fifth example of a connector system shown in FIG. 5A;

FIG. 6A is a schematic perspective top side view of a sixth example of a system for providing breathing assistance having a sixth example of a connector system;

FIG. 6B is a schematic perspective underside view of the system of FIG. 6A;

FIG. 6C is a schematic rear view of the system of FIG. 6A;

FIG. 6D is a schematic perspective view of a fourth example of an oral appliance used in the system of FIG. 6A;

FIG. 6E is a schematic front view of the fourth example of an appliance shown in FIG. 6D;

FIG. 6F is a schematic sectional top view of the fourth example of an oral appliance taken through section G-G of FIG. 6E;

FIG. 6G is a schematic perspective rear view of the sixth example of a connector system shown in FIG. 6A;

FIG. 6H is a schematic sectional perspective view taken through the air inlet chamber of the connector system shown in FIG. 6A;

FIG. 6I is a schematic sectional side view taken through an air outlet chamber of the connector system shown in FIG. 6A;

FIG. 7A is a schematic perspective view of a seventh example of a system for providing breathing assistance having a seventh example of a connector system;

FIG. 7B is a schematic rear view of the system of FIG. 7A;

FIG. 7C is a schematic perspective view of a fifth example of an oral appliance used in the system of FIG. 7A;

FIG. 7D is a schematic rear view of the seventh example of a connector system shown in FIG. 7A; and,

FIG. 7E is a schematic front perspective view of a nasal connecting portion with nasal pillows for use with the connector system of FIG. 7A;

FIG. 8 is schematic block diagram of a system showing air flow and pressure to/from the oropharynx of the user when wearing an oral appliance and CPAP is being administered;

FIG. 9A is a schematic perspective view of an eighth example of a system for providing breathing assistance having an eighth example of a connector system;

FIG. 9B is a schematic perspective view of the system of FIG. 9A with the nasal tube removed;

FIG. 9C is a schematic sectional view of the system of FIG. 9A with the nasal tube removed taken through the centre of the appliance and connector system;

FIG. 9D is a schematic exploded view of the system of FIG. 9A;

FIG. 9E is a rear schematic view of a nasal tube holder of the system of FIG. 9A;

FIG. 9F is a rear schematic view of an adaptor of the system of FIG. 9A; and,

FIG. 9G is a rear schematic view of an interface component of the system of FIG. 9A.

### DETAILED DESCRIPTION OF THE PREFERRED EMBODIMENTS

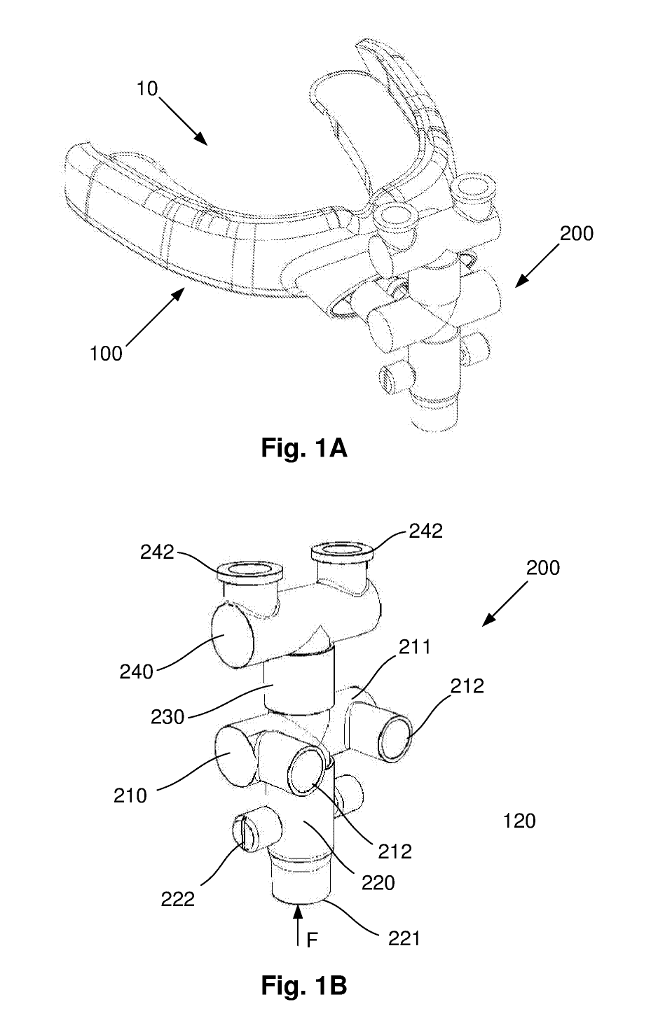

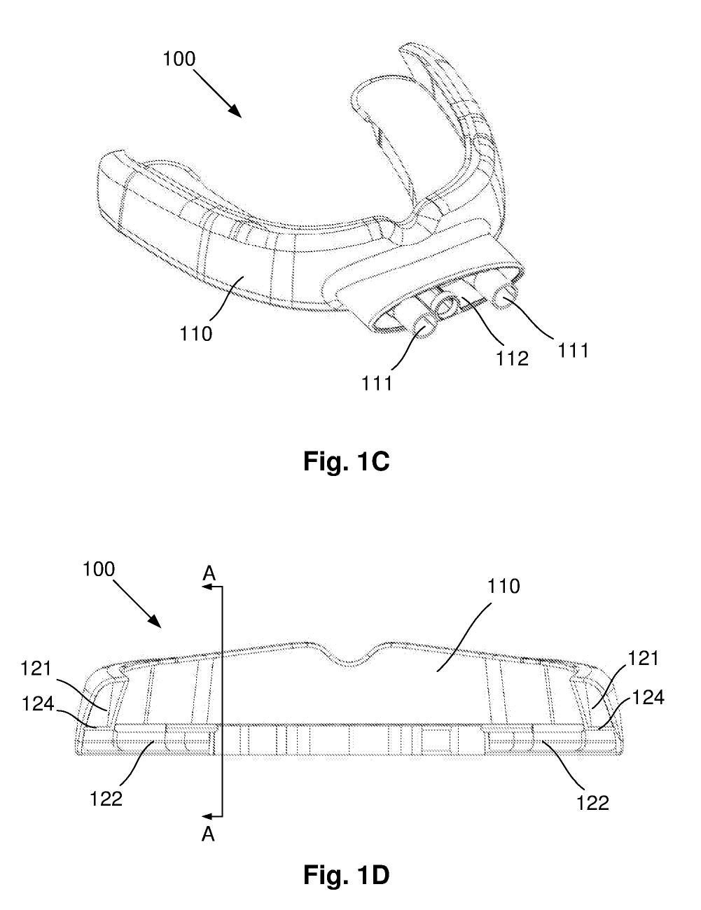

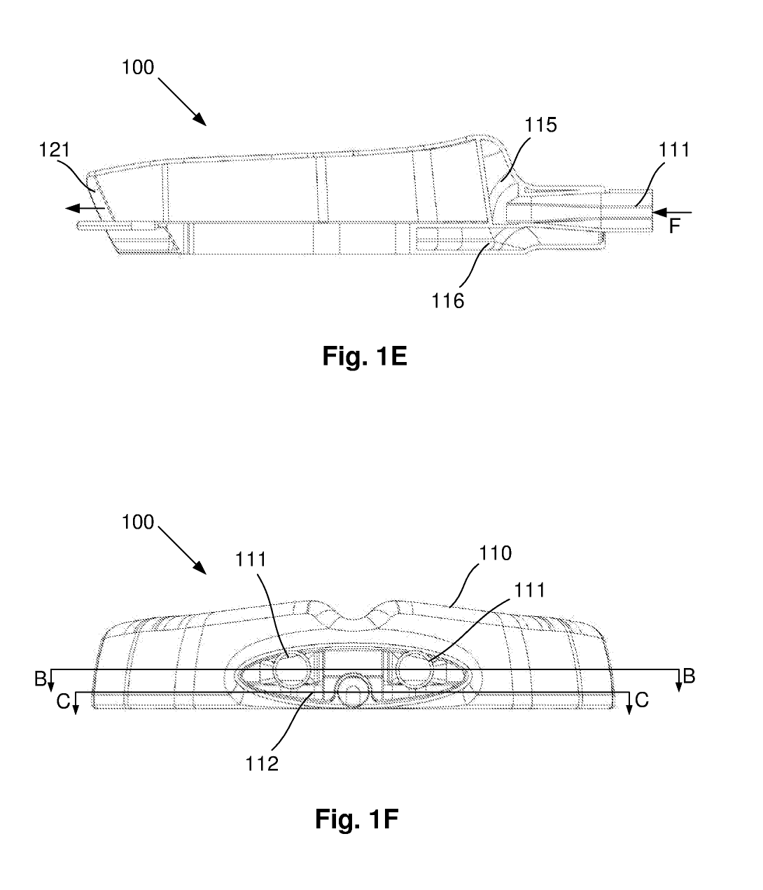

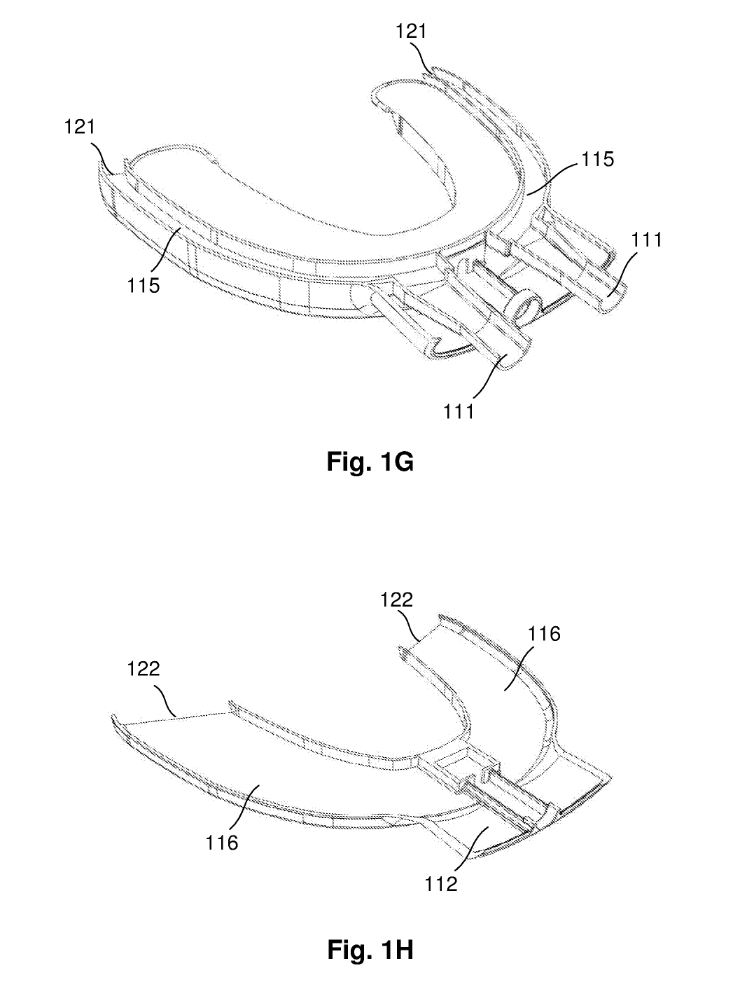

An example of a system for providing breathing assistance to a user will now be described with reference to FIGS. 1A to 1H.

In this example, the system 10 includes an oral appliance 100 including a body 110 for positioning within an oral cavity of the user, the body 110 defining at least one extra-oral opening 111 for allowing airflow between lips of the user in fluid communication with at least one intra-oral opening 121 provided in the oral cavity to direct airflow into and/or out of a posterior region of the oral cavity.

The system further includes at least one of a housing (not shown) connected to the at least one extra-oral opening 111 of the appliance 100, the housing configured to allow natural breathing through the appliance 100, or a connector system 200 for receiving air and/or oxygen from a positive airway pressure (PAP) device, the connector system 200 connected to the at least one extra-oral opening 111 of the appliance 100.

Thus, the oral appliance has at least one first extra-oral opening 111, which can extend beyond the lips, or at least keep the lips apart, to allow airflow therethrough. Air passes through airways defined by channels, and is directed into a posterior region of the mouth through the at least one intra-oral opening 121. By connecting a PAP device such as a continuous positive airway pressure (CPAP) machine to the oral appliance through the connector system, pressure and/or flow can be delivered directly to the oropharynx thereby bypassing obstructions from the nose, soft palate and tongue which can lead to snoring and apnoea events. The system is therefore able to operate at a much lower pressure than a traditional CPAP/mask combination which needs to supply sufficient pressure to open the obstructed airway of the user.

In this manner, the PAP device can be used as a source of pressure and/or flow to supplement natural breathing. For this reason, lower airflow is required from the PAP device compared to a traditional CPAP/mask combination that must provide substantially the entire air flow for the user. Whilst a standard CPAP machine may deliver around 70 litres/minute for breathing at rest, it is expected that flow rates of between 4 to 12 litres/minute would be suitable for a PAP device used in conjunction with the above-described oral appliance. Accordingly, it is to be appreciated that in the above described system, the pressure and flow requirements of a PAP device will be much lower than is currently the case for traditional CPAP/mask systems.

This in turn enables smaller PAP devices to be used, with smaller and less energy intensive pumps that will also reduce noise leading to increased patient comfort and compliance. The PAP devices may also be battery operated making them portable and more convenient for use.

The system may therefore be used for the treatment of sleep apnoea in all severities of patients who require to be treated with positive airway pressure and/or to receive supplemental air and/or oxygen.

The lower pressures and air flow required also overcomes issues with leakage experienced with a mask and the system is essentially maskless which further leads to increased comfort (e.g. no claustrophobia) and compliance.

In other arrangements, a housing may be connected to the at least one extra-oral opening for natural breathing. As will be described in more detail below, one or more sensors such as position, temperature, airflow or pressure sensors may be located within the housing to monitor body or head position, air temperature as well as flow rate and pressure on inhalation and exhalation through the oral appliance. Such sensor data may be used in a feedback control system which in one example may selectively switch on and off a PAP device so that pressure and/or flow is only provided as needed.

A number of further possible features will now be described.

Typically, the at least one extra-oral opening 111 is provided as part of at least one connection portion that at least partially protrudes from the appliance 100 as shown for example in FIG. 1C. In one example, the connection portion is tubular as shown and may have any desired cross-sectional shape, including cylindrical as shown. The connector system 200 will typically include a connector 210 having a body 211 that defines at least one outlet 212 that connects to the at least one connection portion of the oral appliance 100. In this way, a fluid passageway is created between the connector 210 and the oral appliance for delivering air and/or supplemental oxygen thereto from the PAP device.

The body 110 of the oral appliance 100 further defines at least two channels 115, each channel 115 connecting an intra-oral opening 121 to the at least one extra-oral opening 111, each channel 115 passing at least one of at least partially along the buccal cavity and at least partially between the teeth to thereby provide an airway for the user, the airway at least partially bypassing the nasal passage and acting to replicate a healthy nasal passage and pharyngeal space. One such channel arrangement is described in co-pending applications PCT/AU2012/000565 and PCT/AU2015/050144, the contents of which are incorporated herein by reference.

Air from the PAP is delivered to the user through at least one of the nose of the user, the oral appliance via the airway, and, the nose and the oral appliance via the airway. Delivery of air to the nose will be described in further detail below. Whilst typically, air from the PAP supplements natural breathing through the oral appliance, in an alternative example, the user may breathe only through the appliance from air received from the PAP.

However, more often at least one of the extra-oral openings defines a breathing port that is operable to allow natural breathing through the appliance. In one example, the connector body includes a vent in fluid communication with the breathing port to allow natural airflow to and from the appliance through the connector. In at least one example, at least one of the breathing port and vent in the connector body includes at least one of an adjustable or one-way valve for controlling natural breathing. In one example, the valve may provide easy intake of air while controlling exhalation of the user.

In one example, the channel on each side of the appliance is partitioned so as to define a first and second airway. In such an arrangement, the body of the appliance defines a first extra-oral opening in fluid communication with a first intra-oral opening via the first airway, and, a second extra-oral opening in fluid communication with a second intra-oral opening via the second airway. Partitioning the airway allows for increased control of inhalation and exhalation as well as being able to more readily control air pressure and flow supplied to the user as will be discussed further below.

Typically, the connector body is connected to the first extra-oral opening so that air from the PAP device is directed into the appliance through the first airway. The second airway may then be used for at least one of natural breathing (inhalation and exhalation) or just exhalation for example to reduce effort on exhalation and increase comfort.

In one example, the first airway extends along the user's buccal cavity and the second airway extends at least one of between the user's maxillary and mandibular teeth; and, between the user's maxillary and mandibular teeth and along the user's buccal cavity. In this way, the appliance airways are able to direct air into the posterior region of the oral cavity thereby bypassing obstructions from the nose, soft palate and tongue.

As mentioned above, air pressure and/or flow can be delivered separately and/or in conjunction to the respective first and second airways depending on how the system is configured.

The connector system typically includes a connector body defining at least one passageway between an inlet for receiving air from the PAP device and one or more outlets for directing air into the appliance and/or nose of the user. The inlet may be part of the connector body or part of a separate inlet chamber that is connected to the connector body.

In one example, at least one inlet valve is provided downstream of the inlet for regulating pressure and/or flow through the connector. In a more specific example, a first inlet valve is provided to regulate pressure and/or flow into the appliance and a second inlet valve is provided downstream of the first inlet valve for regulating pressure and/or flow into a nasal connecting portion that directs air into the nose of the user. The connector system is therefore highly configurable and/or controllable to direct pressure and/or flow to the appliance and/or nose as required.

Typically, the connector body includes one or more first outlets that connect to one or more corresponding first extra-oral openings of the appliance to allow air from the PAP device into the appliance. In a particular example, the appliance includes a pair of spaced apart first extra-oral openings and the connector body includes a pair of corresponding first outlets for connection therewith for directing air into airways on opposing sides of the appliance.

In addition, the connector body may also include one or more openings that connect to one or more corresponding second extra-oral openings of the appliance to facilitate natural breathing. In such an arrangement, the connector body includes one or more breathing ports in fluid communication with the one or more openings that enable air to be vented from the connector.

In one example, the nasal connecting portion is connected to one or more second outlets of the connector body through which air from the PAP device is received. In one example, a pair of nasal prongs are connected to the nasal connection portion. The nasal connecting portion and/or nasal prongs are at least one of rotatably and/or slidably movable relative to the connector body to enable the nasal connecting portion and nasal prongs to be appropriately positioned for a particular user.

In a further example, nasal pillows are inserted into the nasal prongs. The nasal pillows may be slidably movable with respect to the nasal prongs to provide an adjustment for a particular individual, although this is not required. The nasal pillows are typically made from a thermoplastic material that are custom heat moulded to suit a particular patient. After the nasal pillows are heat set and bent to shape they may also be cut to length. In this way, the nasal pillows are able to be customised to provide optimal comfort and cushioning when inserted into a patient's nostrils. As an alternative to a thermoplastic material, the nasal pillows may be formed from any suitable flexible tubing that is able to be bent and shaped as needed. To assist the flexible tubing in maintaining shape, the wall structure of the tubing may include ductile metal strips or coil that is easily bent but provides additional stiffness to the tubing.

In order to allow for venting from the nose, exhalation ports may be provided in the nasal connecting portion and/or nasal prongs.

In one example, the system also includes the PAP device which may remotely located from the connector system and connected therewith by tubing. Alternatively, due the lower pressure and airflow requirements, the PAP device may be small enough to be mounted on one of the connector system in front of the mouth, the oral appliance or the head, neck or chest of the user. Such a PAP device may be referred to as a ‘micro-pap’.

Further resulting from the lower pressure and airflow requirements, the PAP device may be include a diaphragm air pump instead of an air blower which may further reduce the size and noise produced by the device. The PAP device may also be battery operated making it more portable than traditional CPAP machines that are connected to mains electricity.

As previously mentioned, in an alternative embodiment a housing may be connected to the at least one extra-oral opening for natural breathing. The housing may include a valve/restrictor and/or a heat and moisture exchanger that controls the water and temperature content of the air being inhaled by exchanging heat and moisture with exhaled air. The valve may be used for regulating air flow through the appliance. In one example, this can be used to resist outflow of air from the intra-oral openings to the extra-oral opening. This can assist in regulating breathing and in particular allow for rapid inhalation, whilst ensuring slower exhalation, thereby maintaining a minimum pressure within the system to prevent collapse of the airway and optimising gas exchange within the lungs, for example to minimise the chances of hyperventilation. The valve can be of any suitable form, such as a ball valve, umbrella valve, or the like, and can be adjustable or titratable to ensure that the level of flow control is appropriate to the user.

The housing may additionally and/or alternatively include one or more sensors for measuring an indication of at least one of body or head position, air pressure, air flow rate, temperature, moisture; and, motion.

In this regard, additionally and/or alternatively one or more sensors may be provided in or on the connector body and/or nasal connecting portion for measuring an indication of at least one of body or head position, air pressure, air flow rate, temperature, moisture; and, motion. Typically, the one or more sensors are positioned proximate the one or more breathing and/or exhalation ports in the connector body and/or nasal connecting portion.

In one example, the system further includes at least one electronic processing device coupled to the one or more sensors for determining sensor data indicative of signals from each of the one or more sensors, and causing the sensor data to be wirelessly transmitted to a monitoring device. The monitoring device could be any suitable processing system including for example a remote server or mobile device such as smart phone, tablet or the like. The sensor data may be stored in a remote data store such as a cloud based storage device.

In a further example, the at least one electronic processing device is configured to control at least one of the PAP device and one or more valves in the flow path in accordance with the determined sensor data to thereby regulate pressure and/or flow provided to the user.

In another broad form, there is provided a connector system for connection to an oral appliance for providing breathing assistance to a user, the connector system including a connector having a body connectable to at least one extra-oral opening of the oral appliance, the body defining at least one passageway between an inlet for receiving air from a positive airway pressure (PAP) device and at least one outlet in fluid communication with the inlet for directing the air from the PAP device into at least one of the at least one extra-oral opening of the oral appliance and the nose of the user.

The body of the connector is typically moulded or manufactured using 3D printing.

In a further broad form, there is provided an apparatus for providing breathing assistance, the apparatus including a body for positioning within an oral cavity of a user, the body defining at least one first and second extra-oral opening configured to allow airflow between lips of the user, at least two intra-oral openings provided in the oral cavity to allow air flow into and out of a posterior region of the oral cavity, and, at least two channels, each channel connecting a respective intra-oral opening to at least one of the first and second extra-oral openings and each channel passing at least one of at least partially along the buccal cavity and at least partially between the teeth to thereby provide an airway for the user, the airway at least partially bypassing the nasal passage and acting to replicate a healthy nasal passage and pharyngeal space.

Typically, a first airway is provided between a first extra-oral opening and a first intra-oral opening and a second airway is provided between a second extra-oral opening and a second intra-oral opening, and wherein the first airway is partitioned from the second airway. As previously described partitioning the airway on either side of the apparatus enables inhalation and exhalation to be controlled as well as pressure and/or flow delivered to the user via the apparatus.

Typically, the first airway passes at least partially along the buccal cavity and the second airway passes at least one of at least partially between the user's maxillary and mandibular teeth, and, at least partially between the user's maxillary and mandibular teeth and at least partially along the buccal cavity.

In one example, the first airway and second airway are partitioned so that the first airway is positioned at least one of above the second airway, and, to the outer lateral side of the second airway, although any suitable arrangement may be used.

In one example, the first airway is used during inhalation and the second airway is used during exhalation. This may enable exhalation to be controlled so as to maintain adequate internal pressure but with the minimum amount of effort on inhalation.

Typically, as previously described the at least one first extra-oral opening is connectable to a supply of air from a positive airway pressure (PAP) device. In one example, the at least one first extra-oral opening is connectable to a connector system including a connector having a body in fluid communication with an inlet for receiving air from the PAP device and at least one outlet connected to the at least one first extra-oral opening for supplying the air thereto. The apparatus may further include at least one second extra-oral opening that is connectable to at least one opening in the connector body thereby allowing natural breathing through at least part of the connector.

In one example, the at least one second extra-oral opening is connectable to a sensor housing having at least one sensor for measuring an indication of at least one of head or body position, air pressure, air flow rate, temperature, moisture; and, motion.

In a typical configuration, the at least one first and second extra-oral openings are provided in an extra-oral portion of the apparatus that extends beyond the lips of the user and has a generally elliptic cross section. The first and second extra-oral openings may be provided as part of connection portions that at least partially protrude from the appliance to facilitate simple connection and disconnection with PAP connectors and/or housings (e.g. sensor housings).

In a further broad form, there may be provided a system for providing breathing assistance to a user, the system including an oral appliance including a body for positioning within an oral cavity of the user; and, at least one extra-oral connector coupled to the oral appliance that at least one of modifies an airway, provides an airway, and, monitors an airway.

As described below, PAP connection to the system is optional and in some forms the oral appliance may not need to provide an airway. In this regard, the extra-oral connector may be a nasal connector and the oral appliance may simply provide a means for securely mounting the nasal connector. In this example, the nasal connector may be connected to a PAP device so to provide nasal PAP or alternatively the nasal connector may be provided with a valve/restrictor mechanism (with or without a heat and moisture exchanger) to provide resistance on exhalation thereby enabling control of internal pressure which assists in maintaining an open airway (without PAP).

Alternatively, or in addition to, the at least one extra-oral connector may house at least one sensor for measuring an indication of at least one of head or body position, air pressure, air flow rate, temperature, moisture, and, motion for use in control, sleep testing, compliance analysis and the like. The sensors may be positioned proximate the user's mouth and/or nose for example to monitor air flow and pressure to/from the user's mouth and/or nose. Similarly, the valve to restrict exhalation may be positioned proximate the user's mouth and/or nose so as to control natural breathing from the mouth and/or nose for use in maintaining or supporting an airway through expiratory pressure.

Whilst sensors and/or exhalation valves could be integrated into the system without the oral appliance providing an airway, typically the body of the oral appliance defines at least one extra-oral opening for allowing airflow between lips of the user in fluid communication with at least one intra-oral opening provided in the oral cavity to direct airflow into and/or out of a posterior region of the oral cavity.

In this regard, the body further defines at least two channels, each channel connecting an intra-oral opening to the at least one extra-oral opening, each channel passing at least one of at least partially along the buccal cavity and at least partially between the teeth to thereby provide an airway for the user, the airway at least partially bypassing the nasal passage and acting to replicate a healthy nasal passage and pharyngeal space.

In this regard, the at least one extra-oral connector is connected to the at least one extra-oral opening of the appliance and configured to receive air and/or oxygen from a positive airway pressure (PAP) device. Depending on the construction and configuration of the extra-oral connector, air and/or oxygen from the PAP device may be delivered to the user through at least one of: the nose of the user, the oral appliance via the airway, and, the nose and the oral appliance via the airway.

Delivering PAP through the appliance airway has the ability to reduce the pressure requirements from the PAP due to the low resistance pathway for the pressure to get to the oropharynx as a result of the appliance airway bypassing obstructions from the nose, soft palate and tongue. However, in order to adequately control pressure in the system, in some examples it may be necessary to control exhalation through the appliance airway and/or nose by using a valve/restrictor and/or heat moisture exchange (HME) sponge. Restricting exhalation in this manner enables internal pressure to be controlled and thereby prevents pressure required to maintain an open airway to the oropharynx from escaping the system. An example block diagram showing flow and pressure in such a system is provided in FIG. 8 in which P-Crit represents the critical internal pressure required to support the airway to the oropharynx.

The at least one extra-oral connector may include at least one control valve for directing air and/or oxygen from the PAP device through the connector to at least one of the mouth and/or nose of the user.

The at least one extra-oral connector may also include at least one vent for controlling natural breathing through the mouth and/or nose. The vents could for example be provided proximate the nasal connecting portion or an oral extension portion of the connector which protrudes from the front of the appliance.

Whilst the oral appliance may have a dual airway as described for example in co-pending applications PCT/AU2012/000565 and PCT/AU2015/050144, the channel (or airway) on each side of the oral appliance may be partitioned so as to define a split airway having a first channel that is used for delivering PAP and a second channel that is used for natural breathing. The split airway configuration may also be used for separately controlling pressure and flow through the appliance.

Referring back to FIGS. 1A to 1H, the system 10 for providing breathing assistance to a user shall be described in further detail.

In this example, the oral appliance 100 has a pair of spaced apart first extra-oral openings 111 that protrude from the front of the appliance 100 to facilitate connection with the connector system 200. A second extra-oral opening 112 is provided at the front of the appliance for allowing natural breathing and/or exhalation. Each first-extra oral opening 111 directs air through a channel that defines a first airway 115 to a first intra-oral opening 121 as shown for example in FIG. 1E. The second extra-oral opening 112 directs air through a channel that defines a second airway 116 to a second intra-oral opening 122.

The first and second airways 115, 116 are separated by a partition 124 so that the first airway 115 is disposed above the second airway 122. In use, the first airway 115 extends along the user's buccal cavity and the second airway 116 extends between the user's maxillary and mandibular teeth and along the user's buccal cavity.

The connector system 200 includes a mouth connector 210 including a body 211 for connection to the oral appliance 100. An inlet chamber 220 having an inlet 221 for receiving a flow F of air from a PAP device (not shown) is connected to the mouth connector 210. The mouth connector 210 includes a pair of outlets 212 that connect to the pair of first extra-oral openings 111. In this way, air from the PAP device can be directed into the oral appliance 100 through the first extra-oral openings 111. Air from the PAP is then directed along the first airway 115 to the first intra-oral opening 121 where it is directed into the posterior region of the oral cavity proximate the oropharynx.

The inlet chamber 220 includes an inlet valve 222 for controlling airflow into the connector 210. Any suitable type of valve may be used including for example a butterfly valve.

The mouth connector 210 is further connected to a nasal connecting portion 240 via a restrictor/valve in nasal inlet chamber 230. The nasal connecting portion 240 has a pair of outlets 242 through which air from the PAP device is able to be delivered into the nose of the user. Optionally, nasal pillows or the like may be inserted into the nasal outlets 242 for insertion into the user's nose.

In the above described system 10, air flow and/or pressure is able to be delivered from a PAP device to supplement natural breathing through the appliance 100 which minimises airflow required to be delivered to the user. Furthermore, as air from the PAP is able to be delivered directly to the oropharynx bypassing obstructions from the nose, soft palate and tongue, less pressure is required to maintain an airway compared to a tradition CPAP/mask combination.

Furthermore, the partitioned airway in the appliance enables a separate airway to be provided for exhalation which reduces effort on exhalation and as such increases comfort.

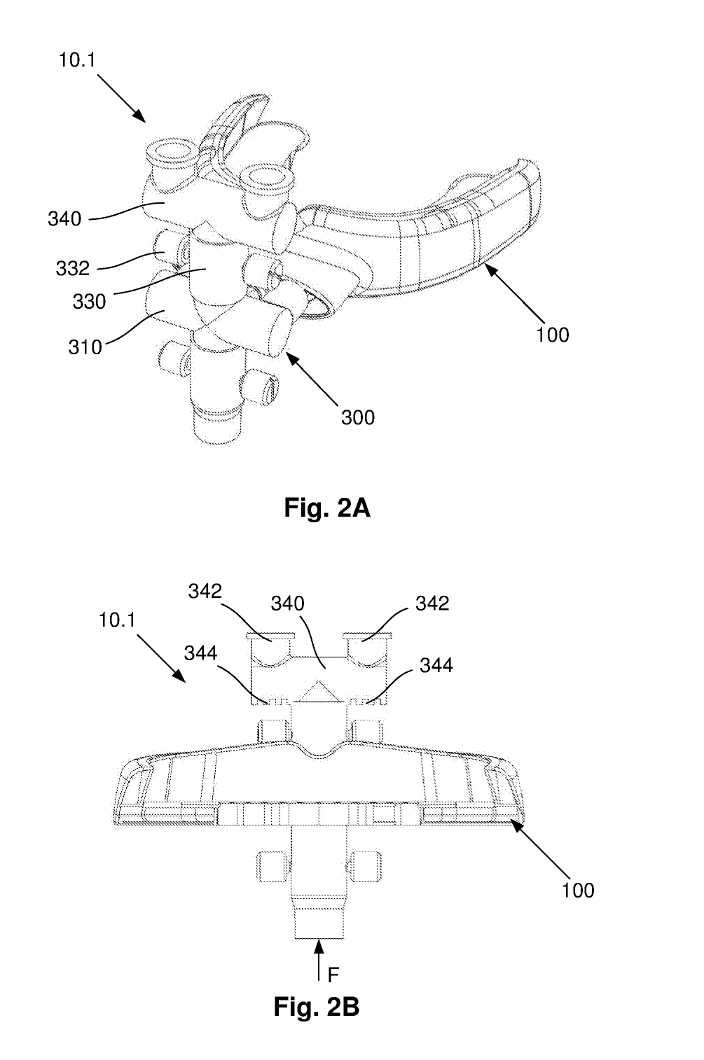

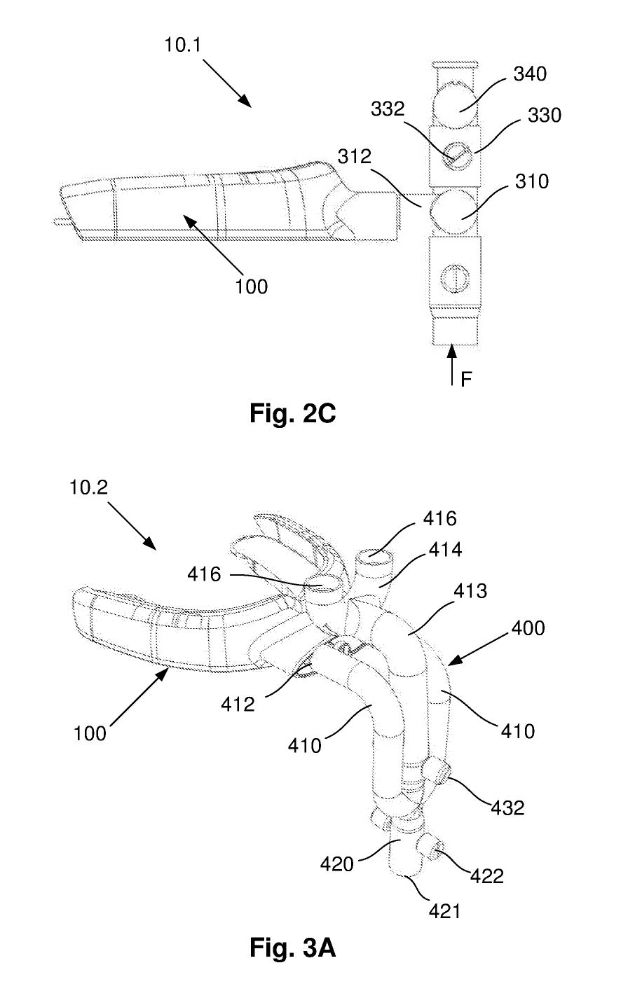

A second example of a system 10.1 for providing breathing assistance to a user shall now be described with reference to FIGS. 2A to 2C. In this example, the system 10.1 includes the oral appliance 100 as previously described connected to a connector system 300. The connector system 300 is similar to connection system 200 and like reference numerals shall be taken to refer to corresponding features as previously described. The connector system 300 additionally includes a valve 332 in a nasal inlet chamber 330 that is adjustable to control air flow into nasal connecting portion 340. The nasal connecting portion 340 further includes a pair of exhalation ports which enable a user to breath out of their nose as well as out through the oral appliance 100.

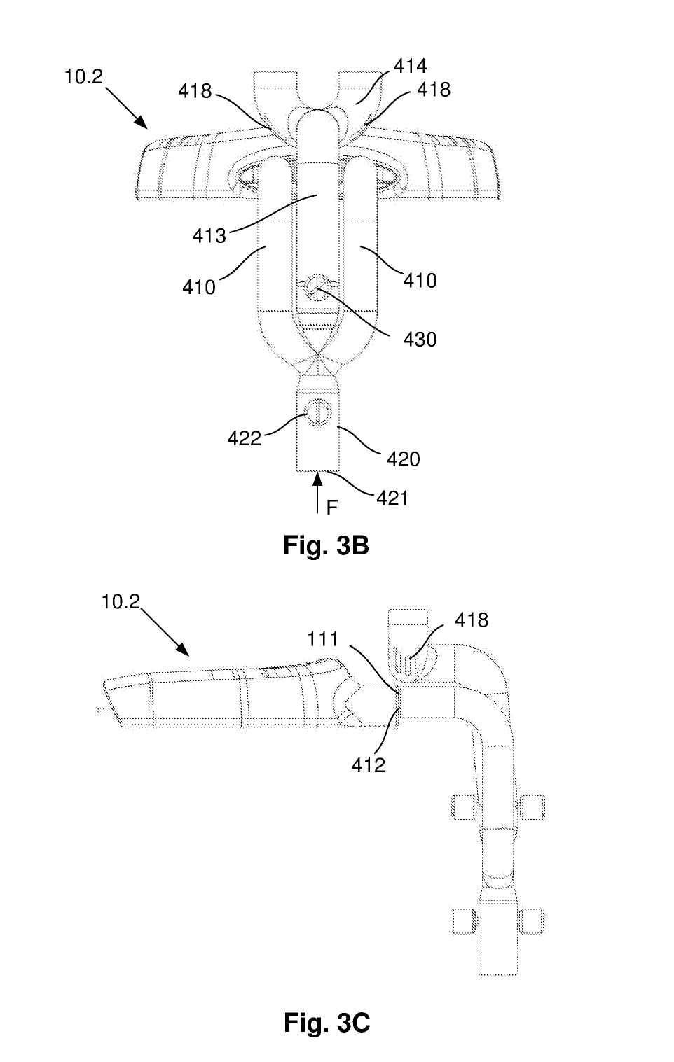

A third example of a system 10.2 for providing breathing assistance to a user shall now be described with reference to FIGS. 3A to 3C. In this example, the system 10.2 includes the oral appliance 100 as previously described connected to a connector system 400. The connector system 400 in this example includes an inlet chamber 420 for receiving air from the PAP device through an inlet 421 that routes flow into a plurality of outlet chambers 410, 413 that are generally right angle tubular members. A pair of outlet chambers 410 each have an outlet 412 that is connected to a respective first extra-oral opening 111 of the appliance 110 for delivering air from the PAP device into the oral appliance 100. The flow into the appliance 100 may be regulated by an inlet valve 422 positioned downstream from the inlet 421 that is able to regulate flow into the outlet chambers 410, 413. A centrally disposed nasal inlet chamber 413 comprises a right angle tubular portion that terminates in an integral nasal connecting portion 414 including a pair of nasal outlets 416 that upwardly project from the nasal inlet chamber 413. The nasal connecting portion 414 further includes a pair of exhalation ports or vents 418 to allow exhalation and/or natural breathing from the nose. In addition, as in the previously described system, the user is able to breathe naturally through a second extra-oral opening at the front of the appliance.

Airflow into nasal inlet chamber 413 is regulated by a nasal inlet valve 432 located downstream of the inlet valve 422. For example, if nasal inlet valve 432 is closed, then all flow from the PAP device is directed into outlet chambers 410 connected to the oral appliance 100.

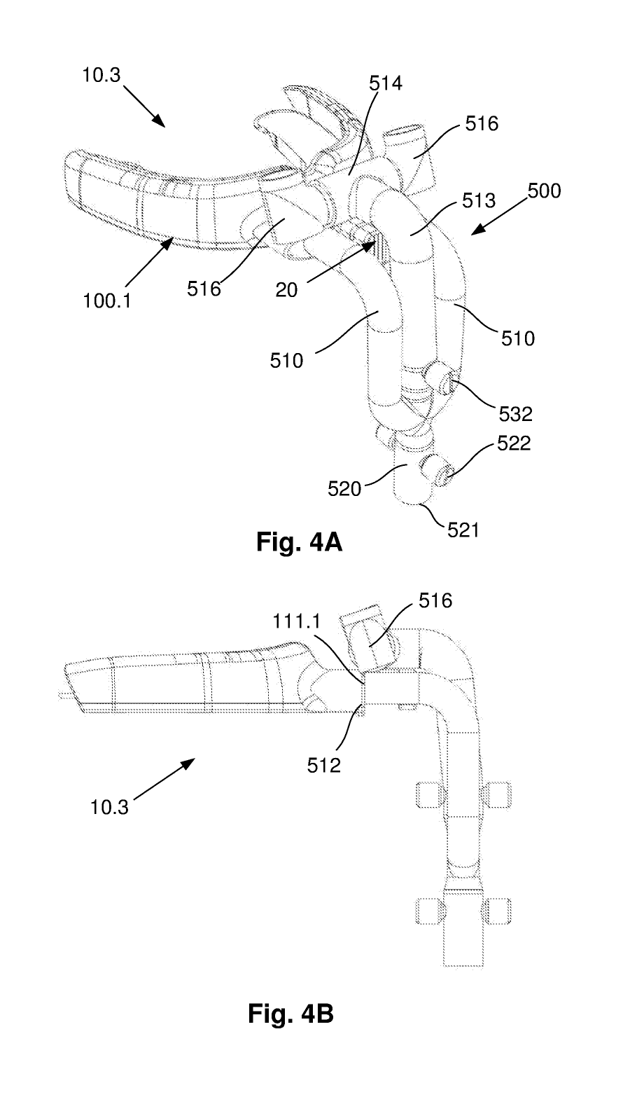

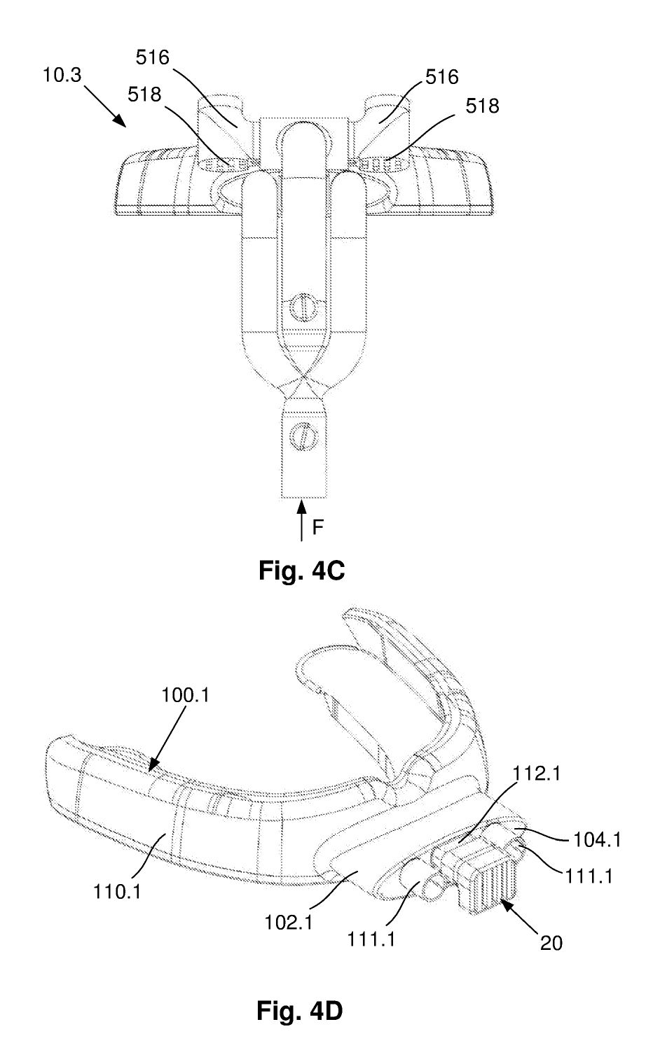

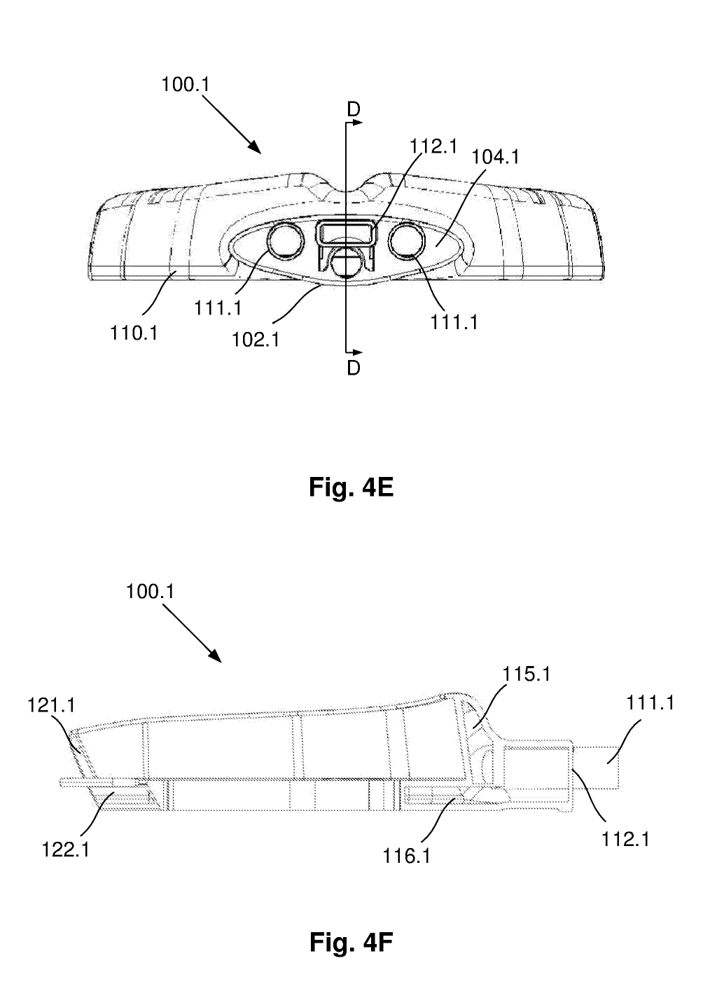

A fourth example of a system 10.3 for providing breathing assistance to a user shall now be described with reference to FIGS. 4A to 4F. In this example, the system 10.3 includes an oral appliance 100.1 having a body 110.1 of similar construction to the previously described appliance 100. However, oral appliance 100.1 has an alternative arrangement of extra-oral openings at the front of the appliance. In this arrangement, the body 110.1 includes an extra-oral portion 102.1 that protrudes from the appliance and in use is disposed between the lips of the user. The extra-oral portion 102.1 has a front surface 104.1 through which first and second extra-oral openings 111.1, 112.1 extend. In this regard, the appliance 100.1 includes a pair of spaced apart first extra-oral openings 111.1 defined by cylindrical connector portions that facilitate connection to the connector system 500. The appliance 100.1 further includes a second extra-oral opening 112.1 defined by a substantially rectangular connector portion to which a housing 20 may be connected as shown in FIG. 4D for example.

In this example, the housing 20 includes an inlet in the form of a vent that allows natural breathing through the second extra-oral openings 112.1. The housing 20 may include a valve/restrictor, for example to control exhalation and/or a heat and moisture exchanger for controlling the water and temperature content of the air being inhaled by exchanging heat and moisture with exhaled air. In other arrangements, one or more sensors such as airflow or pressure sensors may be located within the housing to monitor flow rate and pressure on inhalation and exhalation through the oral appliance.

The connector system 500 is similar to the connector system 400 previously described and like reference numerals shall be taken to refer to corresponding features as previously described. The main difference is that connector system 500 includes an outlet chamber 513 that terminates in a Tee-section nasal connecting portion 514 having a pair of opposed outlets into which adjustable nasal prongs 516 are inserted. The nasal prongs 516 are rotatably and/or slidably connected to the outlets of the nasal connecting portion 514 in order to allow the nasal connection to be configured as appropriate for a particular user. When nasal inlet valve 532 is open, airflow from the PAP device is able to flow into outlet chamber 513 and through nasal prongs 516 into the user's nasal cavity.

In operation, airflow from the PAP device is directed into first extra-oral openings 111.1 and along channels defining an airway 115.1 to intra-oral openings 121.1 whereby air is directed into a posterior region of the oral cavity proximate the oropharynx. Natural breathing is allowed through the second extra-oral opening 112.1 where flow is directed along channels defining a second airway 116.1 to a second intra-oral opening 122.1 as shown for example in FIG. 4F.

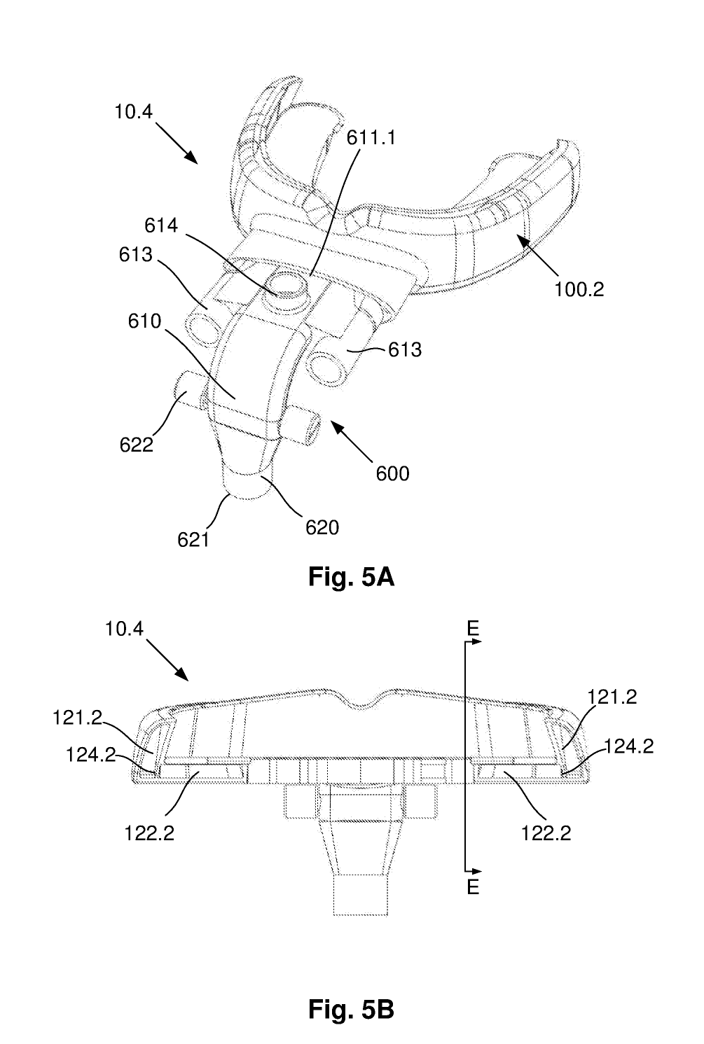

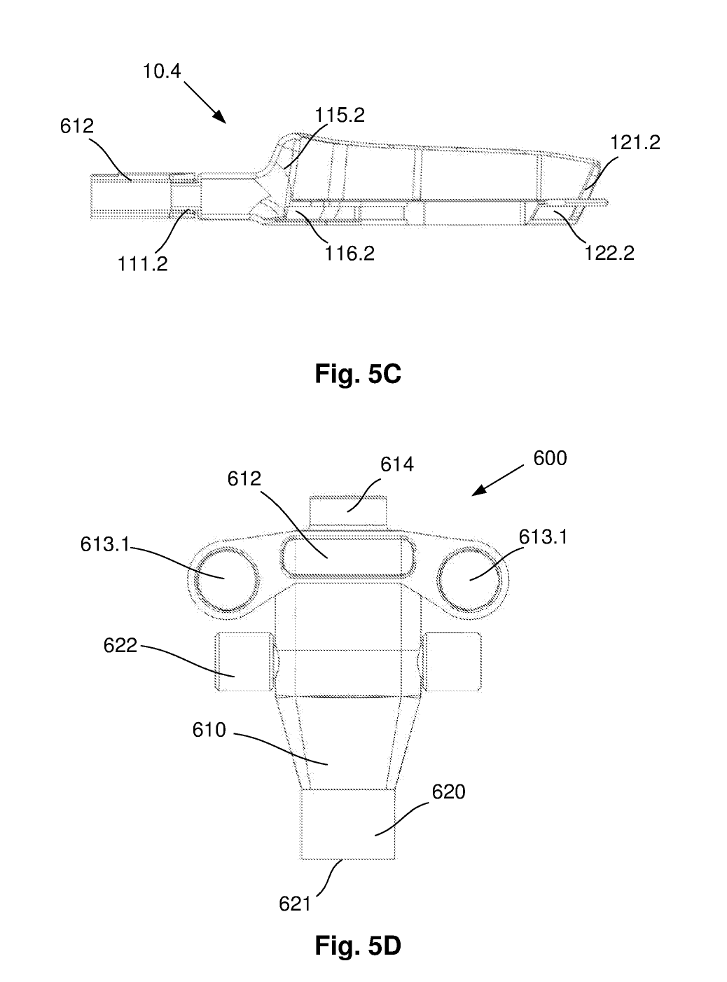

A fifth example of a system 10.4 for providing breathing assistance to a user shall now be described with reference to FIGS. 5A to 5D. In this example, the system 10.4 includes an oral appliance 100.2 having an arrangement of extra-oral openings 111.2, 112.2 as previously described with respect to oral appliance 100.1. A pair of first extra-oral openings 111.2 are in fluid communication with intra-oral openings 121.2 via channels that defines a first airway 115.1. A second extra-oral opening 112.2 is in fluid communication with intra-oral openings 122.2 via channels that define a second airway 122.2. The airways 121.2, 122.2 on each side of the appliance 100.2 are partitioned by a partition 124.2 as shown in FIG. 5B. As a result of this partition, in use, the first airway 115.2 extends along the user's buccal cavity and the second airway 116.2 extends between the user's maxillary and mandibular teeth.

The connector system 600 includes an inlet chamber 620 having an inlet 621 for receiving air from a PAP device. The air is directed through an inlet valve 622 for regulating flow into a connector 610 having a body that terminates in an outlet 612 that is connected to the second extra-oral opening 112.2. Flanking the body of the connector 610 are a pair of cylindrical outlet chambers 613 that have openings 613.1 that connect to the pair of first extra-oral openings 111.2. The connector body further has a nasal inlet connector 614 upstanding from the connector body which permits nasal attachments to be connected thereto (although this is not essential).

In operation, airflow from the PAP device is directed into the second extra-oral opening 112.2 and along second airways 116.2 to intra-oral openings 122.2 whereby air is directed into a posterior region of the oral cavity proximate the oropharynx. Natural breathing and/or exhalation is allowed through the pair of first extra-oral openings 111.2 where flow is directed along first airways 115.2 to or from the first intra-oral openings 121.2 as shown for example in FIG. 5C. Accordingly, it will be appreciated that in this example, the function of the first and second extra-oral openings of the appliance has been reversed from previously described examples.

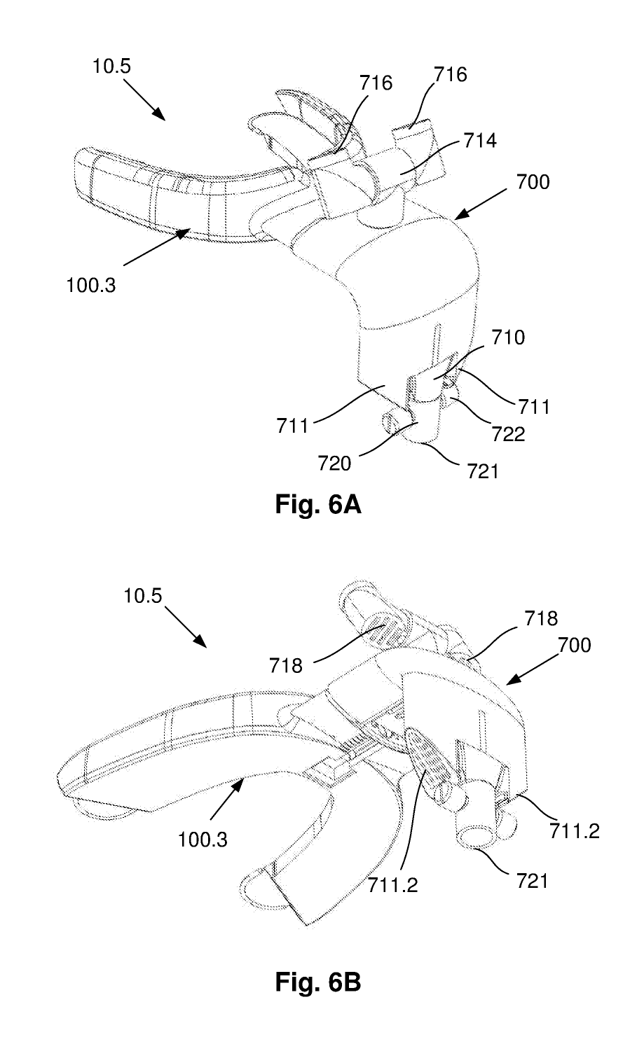

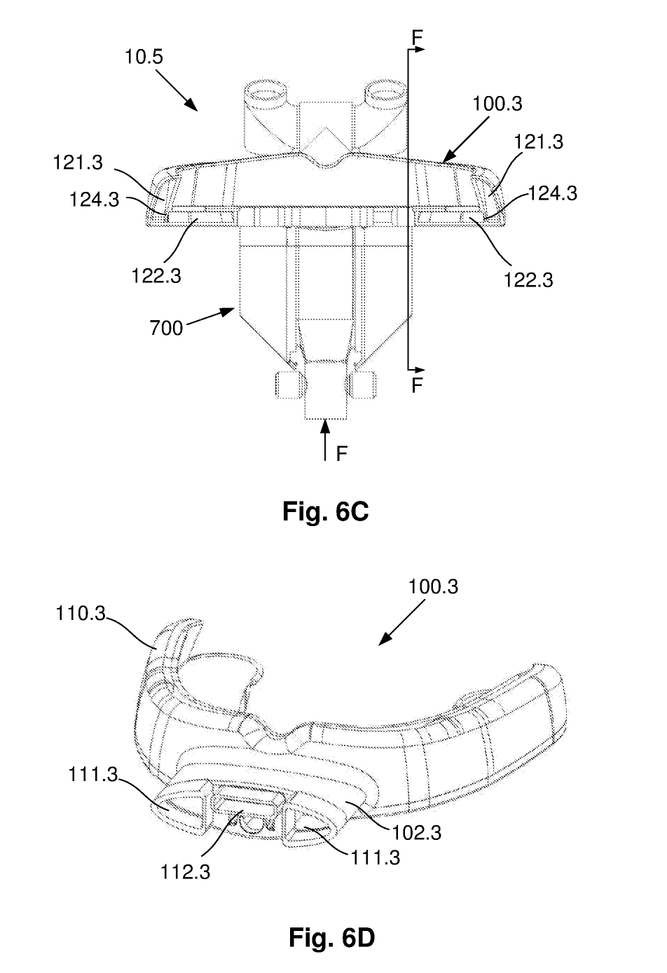

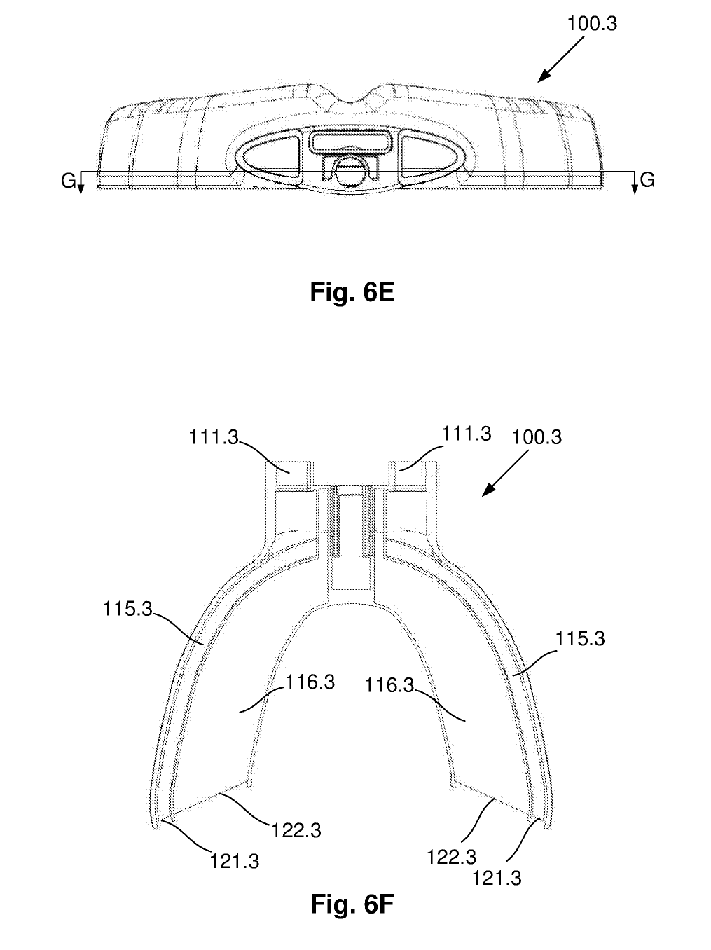

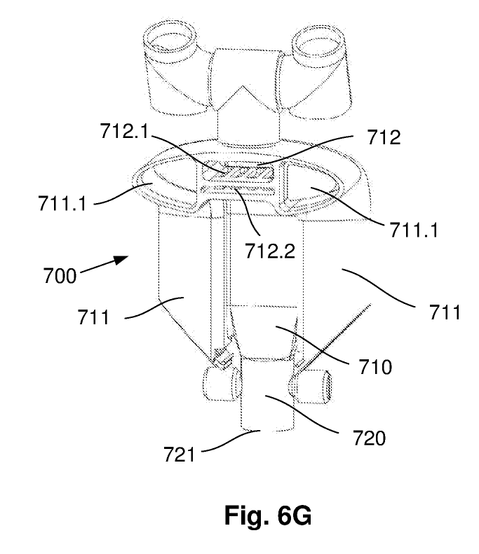

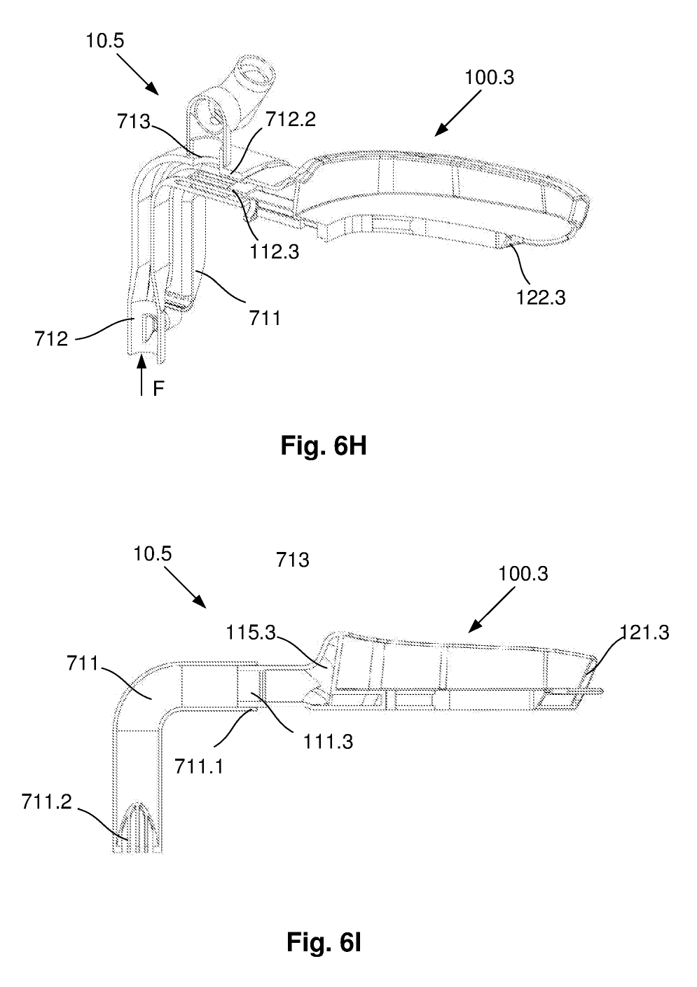

A sixth example of a system 10.5 for providing breathing assistance to a user shall now be described with reference to FIGS. 6A to 6I. In this example, the system 10.5 includes an oral appliance 100.3 having a similar construction to the previously described appliance 100.2. In appliance 100.3 however, the pair of first extra-oral openings 111.3 are part of connector portions having sections defined by a straight inner wall and an arcuate or elliptic outer wall that is substantially complementary to the profile of the extra-oral portion 102.3 as shown in FIG. 6D for example.

The connector system 700 includes an inlet chamber 720 having an inlet 621 for receiving air from a PAP device. The air is directed through an inlet valve 722 for regulating flow into an L-shaped connector 710 having a body that terminates in an outlet 712 (see FIG. 6G) that is connected to the second extra-oral opening 112.3. Integral with and flanking the body of the connector 710 are a pair of L-shaped outlet chambers 711 that have openings 711.1 that connect to the pair of first extra-oral openings 111.3. The connector body further has a nasal connecting portion 714 upstanding from the connector body which permits nasal prongs to be connected thereto as described in previous examples. Although not shown, nasal pillows for entry into the user's nose may be inserted into the nasal prongs. The nasal pillows may be slidably movable with respect to the nasal prongs to provide an adjustment, although this is not required. Alternatively, the nasal pillows may be designed to be bent/universally adjusted to any desired orientation instead of or in addition to the nasal prongs being rotatable and/or slidable.

The connector system 700 provides several natural breathing/exhalation ports as shown for instance in FIG. 6B. In this example, each nasal prong includes an exhalation port or vent 718 through which the user can vent air from their nose. Additionally, each outlet chamber 711 terminates in a port or vent 711.1 to thereby enable the user to naturally breathe or exhale from the oral appliance 100.3. In the example shown, the vents 711.2 are inclined with respect to a lower portion of outlet chamber 711 so as to facilitate greater ease of natural breathing by increasing the vent area.

In this connector system, further vents 712.1, 712.2 are provided proximate opening or outlet 712 that is connected to the second extra-oral opening 112.2. The vents 712.1, 712.2 are spaced apart above one another and in use, permit some air from the PAP device to be vented out of the connector system 700 and alternatively allow additional air intake to and from the connector system 700 which can then be routed into the appliance 100.3 and/or the nose of the user.

In operation, airflow from the PAP device is directed into the second extra-oral opening 112.3 and along second airways 116.3 to intra-oral openings 122.3 whereby air is directed into a posterior region of the oral cavity proximate the oropharynx. Natural breathing and/or exhalation is allowed through the pair of first extra-oral openings 111.3 where flow is directed along first airways 115.3 to or from the first intra-oral openings 121.3 as shown for example in FIGS. 6F and 6I.

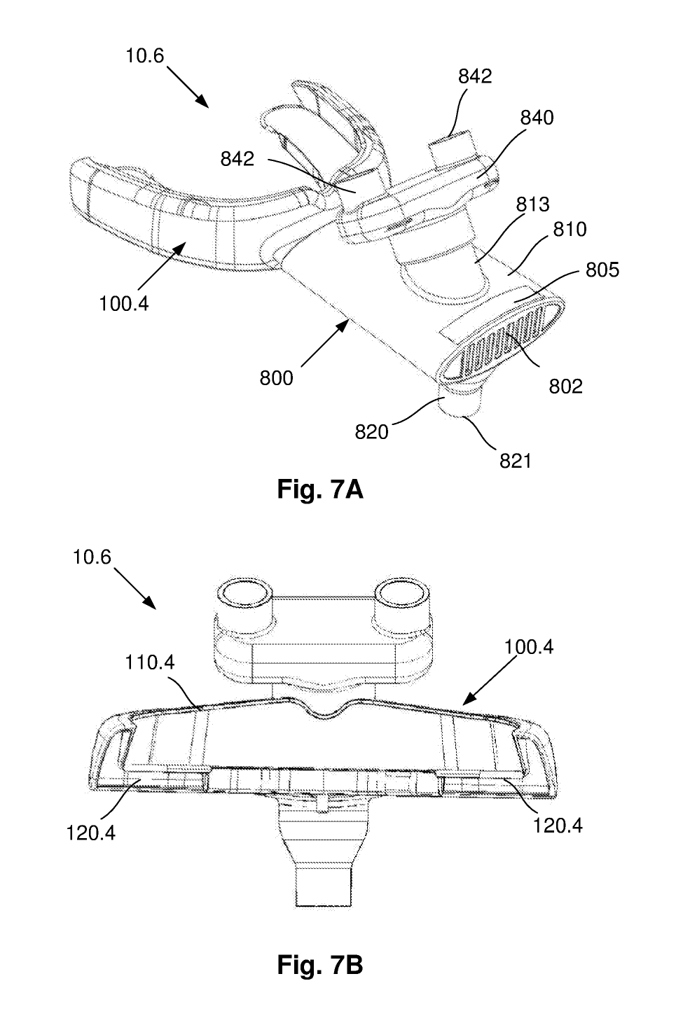

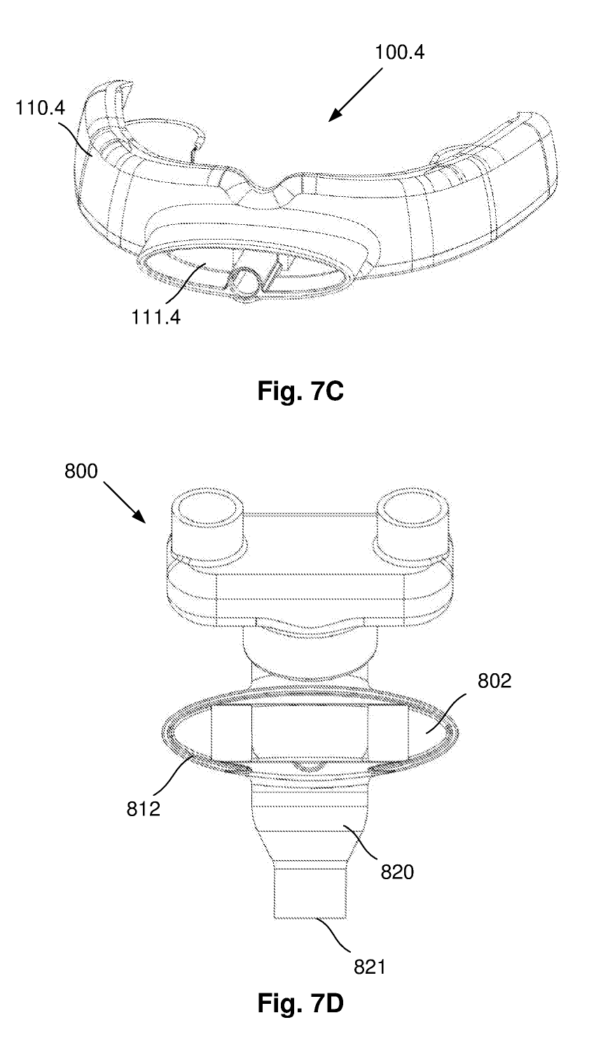

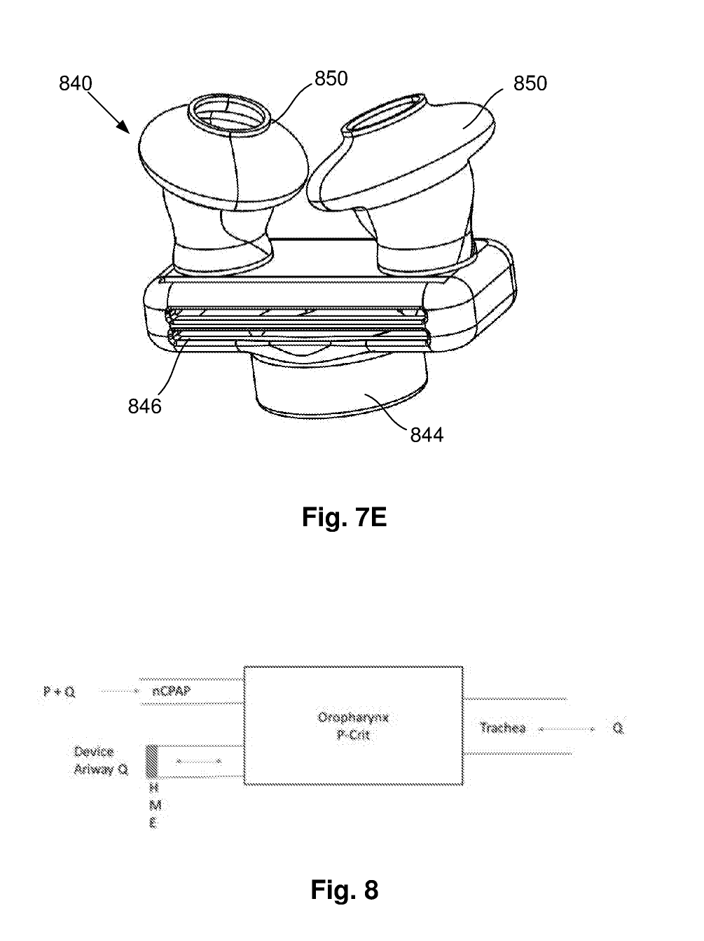

A seventh example of a system 10.6 for providing breathing assistance to a user shall now be described with reference to FIGS. 7A to 7D. In this example, the system 10.6 includes an oral appliance 100.4 substantially as described in co-pending Australian Patent Application No. 2016901171 having a body 110.4 defining a single extra-oral opening 111.4 in fluid communication with respective intra-oral openings 120.4 via channels which define a dual airway for directing airflow to the posterior region of the oral cavity. The respective airways extend at least partially along the buccal cavity and at least partially between the teeth of the user in use.

The connector system 800 includes a connector 810 having a body of elliptic section with an opening 812 that is complementary to the profile of the extra-oral opening 111.4 of the appliance 100.4 for connection thereto. Depending downwardly from the body 810 is an inlet chamber 820 having an inlet 821 for allowing air from a PAP device into the inlet chamber 820. The inlet chamber 820 extends through the connector body 810 and transitions into a nasal inlet chamber 813 that projects away from the body 810. In the example shown, the nasal inlet chamber 813 is inclined relative to a direction of elongation of the connector body 810. A nasal connecting portion 840 is connected to the nasal inlet chamber 813, the nasal connecting portion 840 having a pair of outlets 842 for directing air from the PAP device into the nasal cavity of the user.

At a distal end of the connector body 810 is a breathing port or vent 802 having an adjustable valve for allowing easy intake of air and controlled exhalation through the appliance 100.4. Additionally, a heat and moisture exchanger (HME) 805 may also be provided for controlling the water and temperature content of the air being inhaled by exchanging heat and moisture with exhaled air. In one example, the heat and moisture exchanger may also act as the one-way valve. In such an arrangement, the heat and moisture exchanger may comprise a flap of material that is hingedly connected inside the connector body 810 proximate the vent 802. In use, as the patient breathes in, the flap pivots away from the vent 802 to allow airflow into the appliance 100.4 When the user exhales, the flap pivots back towards the vent to substantially close the flow path thereby creating resistance upon exhalation. The level of resistance to exhalation may be controlled in any suitable manner including by providing one or more holes in the flap of HME material to provide a flow path for the expired air. It is to be understood that a one-way valve formed of HME material may also be implemented in any of the previously described examples.

Typically, the user is able to breathe naturally through the appliance 100.4 with air flow travelling from vent 802 (through valve and/or HME) through the connector body 810 into the appliance 100.4 then through extra-oral opening 111.4 and along the airway to intra-oral openings 120.4 where it is directed into a posterior region of the oral cavity. Meanwhile, nasal PAP can be delivered into the user's nose from the PAP device via the flow path formed by the tubing that extends through the connector body. In this regard, it is to be understood that in this example PAP is not delivered through the oral appliance 100.4 and only to the nasal cavity of the user. The connector body 810 is therefore used as a convenient means to secure the nasal PAP connector system to the oral appliance 100.4.

In FIG. 7E, there is shown an example of a nasal connecting portion 840 having a pair of nasal pillows 850 configured for insertion into the user's nostrils. The nasal pillows 850 are typically made from a thermoplastic material that are custom heat moulded to suit a particular patient. After the nasal pillows 850 are heat set and bent to shape they may then be cut to appropriate length. In this way, the nasal pillows 850 are able to be customised to provide optimal comfort and cushioning when inserted into a user's nostrils. As an alternative to a thermoplastic material, the nasal pillows may be formed from any suitable flexible tubing that is able to be bent and shaped as needed. To assist the flexible tubing in maintaining shape, the wall structure of the tubing may include ductile metal strips or coil that is easily bent but provides additional stiffness to the tubing. The nasal pillows 850 may be sleeved over the outlets 842 shown in FIG. 7A. The nasal connecting portion 840 further includes an adaptor portion 844 for engagement with the nasal inlet chamber 813. One or more vents 846 may also be provided in the body of the nasal connecting portion 840.

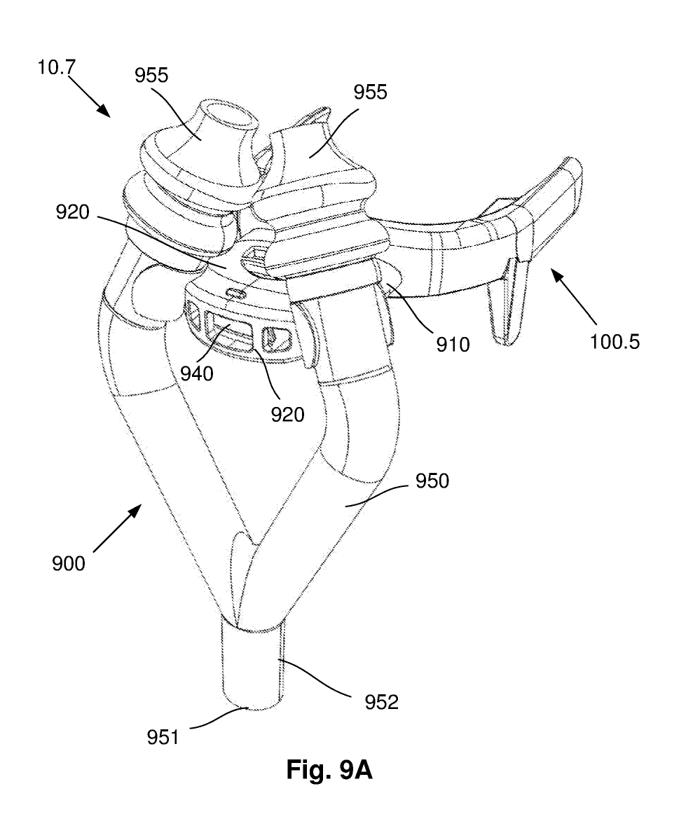

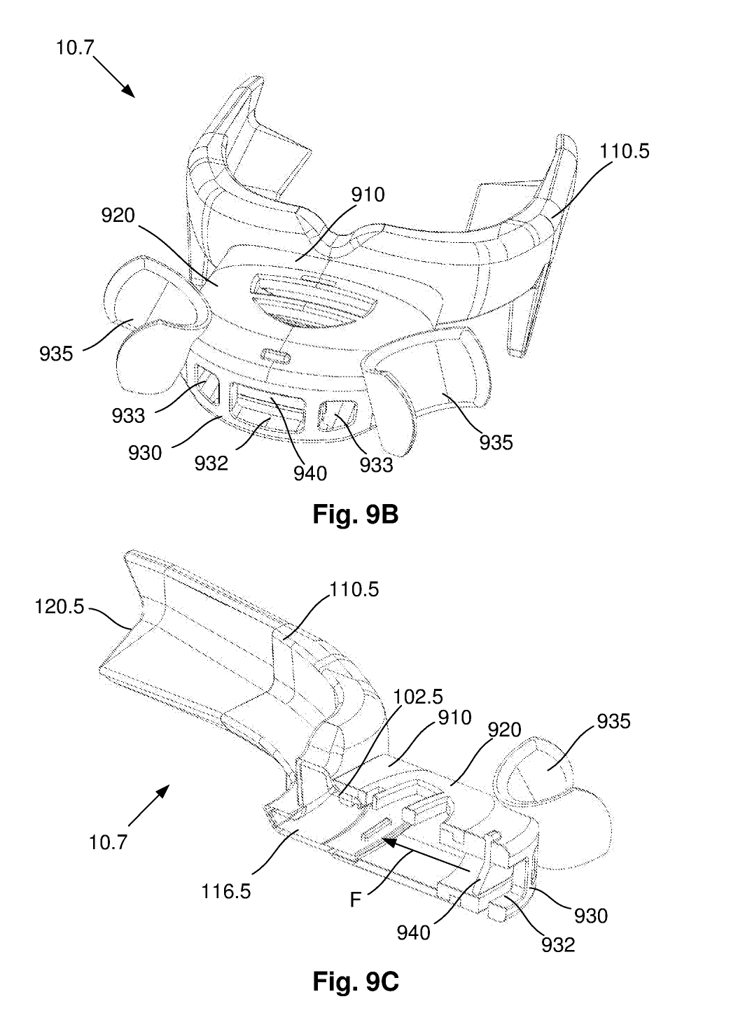

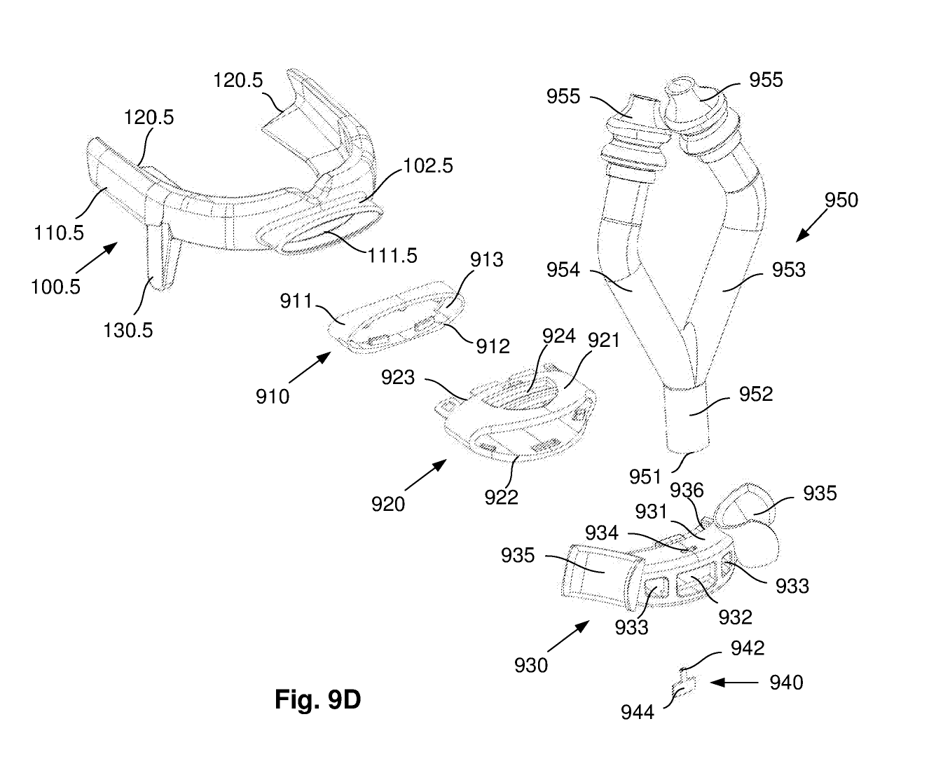

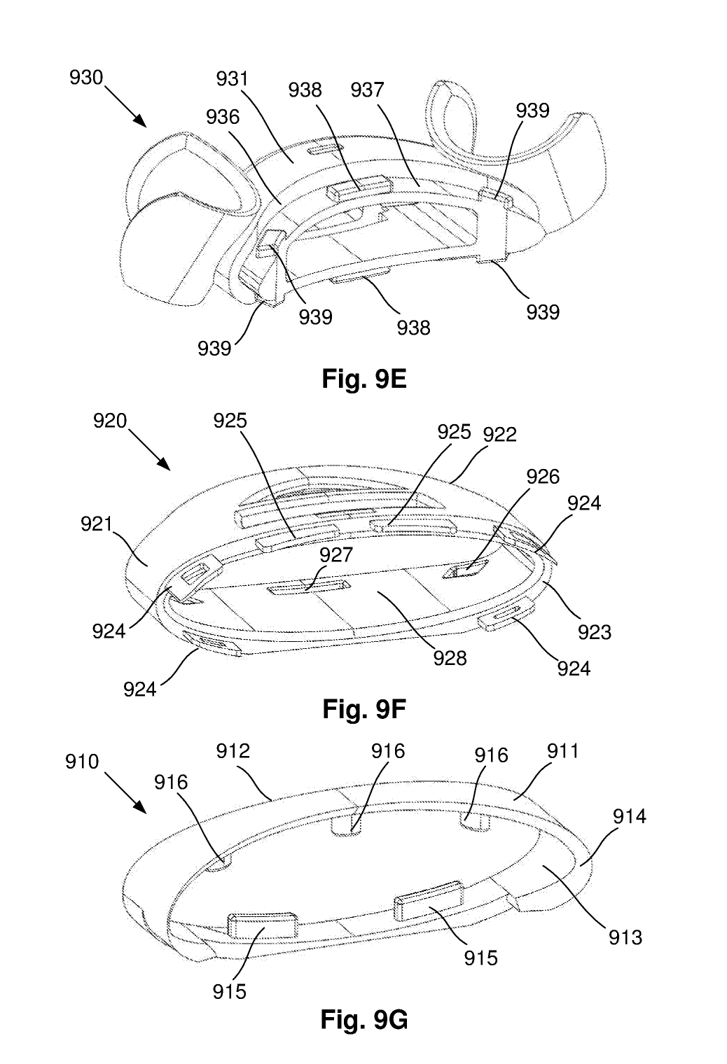

An eighth example of a system 10.7 for providing breathing assistance to a user shall now be described with reference to FIGS. 9A to 9G. In this example, the system 10.7 includes an oral appliance 100.5 substantially as described in International Patent Application No. PCT/AU2017/050271 having a body 110.5 defining a single extra-oral opening 111.5 in fluid communication with respective intra-oral openings 120.5 via channels which define a dual airway for directing airflow to the posterior region of the oral cavity. The respective airways extend at least partially along the buccal cavity and at least partially between the teeth of the user in use.

The appliance 100.5 shown in this example is an adjustably configurable appliance that may for example allow the position of a user's lower jaw to be adjusted relative to the position of the upper jaw. It is to be appreciated that the body 110.5 shown in this example forms an upper body of the adjustable appliance and that a lower body to which the upper body is adjustably mounted is not shown (as the airway is provided in the upper body only). A pair of wings 130.5 extend downwardly from the body 110.5 towards the lower body in use. A pair of blocks are adjustably mounted to the lower body to allow the blocks to be moved in alongitudinal direction and wherein in use the wings 130.5 engage the blocks to constrain relative longitudinal movement of the bodies to thereby selectively advance the mandibular teeth relative to the maxillary teeth.

In this example, a connector system 900 is attached to the oral appliance 100.5. The connector system 900 is a multi-part assembly which permits oral breathing through the appliance 100.5 as well as delivery of PAP to the nose of the user. The connector system 900 includes an appliance interface 910 which mounts to the extra-oral portion 102.5 (i.e. duckbill) of the appliance 100.5. An adaptor 920 then plugs into the front of the interface 910 and a nasal tube holder 930 for supporting a nasal tube 950 plugs into the front of the adaptor 920.

In addition to providing supports 935 for the nasal tube 950, the nasal tube holder 930 also provides openings 932, 933 in a front surface thereof in fluid communication with the extra-oral opening 111.5 of the appliance 100.5 to thereby permit oral breathing through the connector system 900. In this example, a one-way valve 940 is also positioned behind opening 932 of the nasal tube holder 930 to control oral breathing by allowing easy inhalation and controlled exhalation. The adaptor 920 may also include one or more openings or vents 924 provided in an upper surface thereof for venting air and/or allowing one or more appliance monitoring devices such as a pressure sensor, humidity sensor, movement sensor, temperature sensor and moisture sensor to be mounted therein so as to be positioned in the flow path.

The nasal tube 950 includes an inlet chamber 952 having an inlet 951 that may be connected to a PAP device. The inlet chamber 952 branches off into two tubes 953, 954 which lead to respective outlets defined by pillows 955 that are inserted into the user's nostrils. In this manner, nasal PAP may be provided to the user to provide a supplemental source of air or oxygen to assist breathing. The nasal tube 950 is conveniently supported by the nasal tube holder 930 which is coupled to the oral appliance 100.5 via the adaptor 920 and interface 910 members. In this regard, the nasal tube supports 935 are typically attached to or integral with the body 931 of the nasal tube holder 930. In this example, the supports 935 are in the form of resiliently deformable clips that clip around the respective tubes 953, 954 of the nasal tube 950.

Referring now to FIG. 9C, there is shown a sectional view through the centre of the system 10.7 showing flow F through the connector 900 during oral breathing. During inhalation, air flows through openings 932, 933 in the nasal tube holder 930. The valve 940 pivots away from opening 932 and allows easy intake of air. Air then flows through the cavity of the adaptor 920 and into the appliance 100.5 via the extra-oral opening 111.5 provided in the duckbill 102.5. The air then flows along channels 116.5 of the appliance 100.5 and exits via the intra-oral openings 120.5 into the posterior region of the user's oral cavity. During exhalation, air follows the reverse pathway but will cause one-way valve 940 to pivot towards the opening 932 and thereby at least partially block the opening 932 to create resistance upon exhalation. In the example shown, the one-way valve 940 includes an upper section 942 that is removably coupled within a slot 934 provided through an upper surface of the nasal tube holder 930. A lower section or flap 944 downwardly depends from the upper section 942 and is configured to hinge or pivot with respect to the upper section 942. In order to obtain the desired cut-off of flow upon exhalation, the flap 944 is typically rearwardly inclined with respect to the upper section 934 such that during exhalation the flap 944 is oriented in a substantially vertical manner behind the opening 932.

The assembly of the above-described connector system 900 will now be described in further detail. Firstly, interface member 910 is engaged over the duckbill 102.5 of the oral appliance 100.5. The interface 910 has an elliptically shaped body 911 including an internal surface 931 that is profiled to fit around the exterior surface of the duckbill 102.5. The interface is pressed onto the duckbill 102.5 so that the rear surface 914 of the interface 910 is substantially flush with a front surface of the body 110.5 of the appliance 100.5. The interface 910 is typically coupled to the duckbill 102.5 via a number of resiliently deformable tabs 915, 916 which protrude away internal surface 913. The tabs 915, 916 snap into corresponding apertures (not shown) provided in the duckbill 102.5 in order to lock the interface 910 in position.

The adaptor 920 is then plugged into the interface 910 and located in position via a number of tabs 924, 925 which protrude away from a rear surface 923 of the body 921 of the adaptor 920 and locate into corresponding apertures or slots (not shown) formed in a front surface 912 of the interface 910.

Finally, the nasal tube holder 930 is plugged into the adaptor 920 via a number of tabs 938, 939 positioned around a recessed portion 937 of the rear of body 931 that are received in corresponding apertures 926, 927 disposed in an inner surface 928 of the adaptor 920. When connected, rear surface 936 of the nasal tube holder 930 is in abutment with the front surface 922 of the adaptor 920.

In one or more of the above described examples, sensors may be incorporated into the connector system and/or housing to monitor parameters such as body or head position, air pressure, air flow rate, temperature, moisture, and, motion. The sensors may be positioned so as to measure these parameters on inhalation and/or exhalation from the nose and the oral appliance. For example, air flow and pressure sensors may be mounted proximate vents or natural breathing ports provided in the connector system. In one example, airflow and/or pressure sensors can be made from polyvinylidene fluoride (PVDF) or from dedicated air flow chips. Wiring for the airflow and/or pressure sensors can be embedded in or coiled around the PAP hose to the central processing unit (CPU)/power supply.

Typically, the system will further include at least one electronic processing device coupled to the one or more sensors for determining sensor data indicative of signals from each of the one or more sensors and controlling at least one of the PAP device and one or more valves in the flow path in accordance with the determined sensor data to thereby regulate pressure and/or flow provided to the user. As the oral appliance has an airway for breathing naturally, if airflow and pressure are sufficient then the PAP device may be switched off as supplemental airflow and/or pressure is not required. Selective use of the PAP device on an as needed basis will further save battery power (in the case of a battery operated device) and reduce noise.

The at least one electronic processing device may further cause the sensor data to be wirelessly transmitted to a monitoring device such as a remote server or client device such as a smart phone or tablet. In practice the monitoring device can communicate with the at least one electronic processing device via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, or the like. The sensor data may be stored in a remote data store, such as in a cloud based storage device for further analysis such as compliance monitoring.

Accordingly, in at least one example, the systems, oral appliances and connector systems described herein enable PAP treatment to be administered to a user suffering from snoring or sleep apnoea using a PAP device having a much lower airflow and pressure than traditional CPAP/mask systems. This is due at least in part to the fact that air from the PAP device can be delivered directly to the oropharynx thereby bypassing obstructions from the nose, soft palate and tongue. The system enables a PAP device to be used as a source of pressure and/or flow to supplement natural breathing. For this reason, airflow from the PAP is much less than that required in traditional PAP systems where the user relies on the PAP system for breathing. This in turn enables smaller PAP devices to be used, with smaller and less energy intensive pumps that will also reduce noise leading to increased patient comfort and compliance. The PAP devices may also be battery operated making them portable and more convenient for use. The system is also essentially maskless which further leads to increased comfort and compliance compared to traditional systems.

In at least one further example, where there is no PAP connection, the system may couple a valve/restrictor mechanism (with or without a heat and moisture exchanger) either directly or indirectly to an oral appliance for controlling inhalation/exhalation. In particular, the valve may allow normal or easier inhalation while creating resistance on exhalation which increases internal pressure and assists in maintaining and/or stabilising an airway so as to assist breathing. In a further example, the system may incorporate one or more sensors such as position, temperature, airflow or pressure sensors to monitor body or head position, air temperature as well as flow rate and pressure to/from the mouth and/or nose of the patient for use in control, sleep testing, compliance and the like.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

Persons skilled in the art will appreciate that numerous variations and modifications will become apparent. All such variations and modifications which become apparent to persons skilled in the art, should be considered to fall within the spirit and scope that the invention broadly appearing before described.

The claims defining the invention are as follows:

1) A system for providing breathing assistance to a user, the system including:
a) an oral appliance including a body for positioning within an oral cavity of the user, the body defining at least one extra-oral opening for allowing airflow between lips of the user in fluid communication with at least one intra-oral opening provided in the oral cavity to direct airflow into and/or out of a posterior region of the oral cavity; and,
b) at least one of:
i) a housing connected to the at least one extra-oral opening of the appliance, the housing configured to allow natural breathing through the appliance; and,
ii) a connector system for receiving air from a positive airway pressure (PAP) device, the connector system connected to the at least one extra-oral opening of the appliance.

2) The system according to claim 1, wherein the at least one extra-oral opening is provided as part of at least one connection portion that at least partially protrudes from the appliance.

3) The system according to claim 2, wherein the at least one connection portion is tubular.

4) The system according to claim 2 or claim 3, wherein the connector system includes a connector having a body that defines at least one outlet that connects to the at least one connection portion of the oral appliance.

5) The system according to any one of the preceding claims, wherein the body further defines at least two channels, each channel connecting an intra-oral opening to the at least one extra-oral opening, each channel passing at least one of at least partially along the buccal cavity and at least partially between the teeth to thereby provide an airway for the user, the airway at least partially bypassing the nasal passage and acting to replicate a healthy nasal passage and pharyngeal space.

6) The system according to claim 4, wherein air from the PAP is delivered to the user through at least one of:
a) the nose of the user;
b) the oral appliance via the airway; and,
c) the nose and the oral appliance via the airway.

7) The system according to claim 6, wherein at least one of the extra-oral openings defines a breathing port that is operable to allow natural breathing through the appliance.

8) The system according to claim 7, wherein the connector body includes a vent in fluid communication with the breathing port.

9) The system according to claim 7 or claim 8, wherein at least one of the breathing port and vent in the connector body includes at least one of an adjustable or one-way valve for controlling natural breathing.

10) The system according to any one of claims 5 to 9, wherein the channel on each side of the appliance is partitioned so as to define a first and second airway.

11) The system according to claim 10, wherein the body of the appliance defines:
a) a first extra-oral opening in fluid communication with a first intra-oral opening via the first airway; and,
b) a second extra-oral opening in fluid communication with a second intra-oral opening via the second airway.

12) The system according to claim 11, wherein the connector body is connected to the first extra-oral opening so that air from the PAP device is directed into the appliance through the first airway.

13) The system according to claim 12, wherein the second airway is used for at least one of:
a) natural breathing; and,
b) exhalation.

14) The system according to any one of claims 11 to 13, wherein the first airway extends along the user's buccal cavity and the second airway extends at least one of:
a) between the user's maxillary and mandibular teeth; and,
b) between the user's maxillary and mandibular teeth and along the user's buccal cavity.

15) The system according to claim 11 or claim 12, wherein air pressure and/or flow can be delivered separately and/or in conjunction to the respective first and second airways.

16) The system according to claim 1, wherein the connector system includes a connector body defining at least one passageway between an inlet for receiving air from the PAP device and one or more outlets for directing air into the appliance and/or nose of the user.

17) The system according to claim 16, wherein at least one inlet valve is provided downstream of the inlet for regulating pressure and/or flow through the connector.

18) The system according to claim 17, wherein a first inlet valve is provided to regulate pressure and/or flow into the appliance and a second inlet valve is provided downstream of the first inlet valve for regulating pressure and/or flow into a nasal connecting portion that directs air into the nose of the user.

19) The system according to any one of claims 16 to 18, wherein the connector body includes one or more first outlets that connect to one or more corresponding first extra-oral openings of the appliance to allow air from the PAP device into the appliance.

20) The system according to claim 19, wherein the connector body includes one or more openings that connect to one or more corresponding second extra-oral openings of the appliance to facilitate natural breathing.

21) The system according to claim 20, wherein the connector body includes one or more breathing ports in fluid communication with the one or more openings.

22) The system according to any one of claims 18 to 21, wherein the nasal connecting portion is connected to one or more second outlets of the connector body.

23) The system according to claim 22, wherein nasal prongs are connected to the nasal connection portion.

24) The system according to claim 23, wherein the nasal connecting portion and/or nasal prongs are at least one of rotatably and/or slidably movable relative to the connector body.

25) The system according to claim 23 or claim 24, wherein nasal pillows are inserted into the nasal prongs.

26) The system according to claim 25, wherein the nasal pillows are slidably movable with respect to the nasal prongs.

27) The system according to any one of claims 23 to 26, wherein exhalation ports are provided in the nasal connecting portion and/or nasal prongs.

28) The system according to any one of the preceding claims, further including a PAP device.

29) The system according to claim 28, wherein the PAP device is battery operated.

30) The system according to claim 29, wherein the PAP device includes a diaphragm air pump.

31) The system according to any one of claims 28 to 30, wherein the PAP device is mounted on:
a) the connector system in front of the mouth;
b) the oral appliance;
c) the arm, head, neck or chest of the user.

32) The system according to claim 27, wherein one or more sensors are provided in or on the connector body and/or nasal connecting portion for measuring an indication of at least one of:
a) body or head position;
b) air pressure;
c) air flow rate;
d) temperature;
e) moisture; and,
f) motion.

33) The system according to claim 32, wherein the one or more sensors are positioned proximate the one or more breathing and/or exhalation ports in the connector body and/or nasal connecting portion.

34) The system according to claim 1, wherein the housing connected to the at least one extra-oral opening that permits natural breathing includes at least one of:
a) a valve/restrictor; and,
b) a heat and moisture exchanger.

35) The system according to claim 34, wherein the housing includes one or more sensors for measuring an indication of at least one of:
a) body or head position;
b) air pressure;
c) air flow rate;
d) temperature;
e) moisture; and,
f) motion.

36) The system according to any one of claims 32 to 35, wherein the system further includes at least one electronic processing device coupled to the one or more sensors for:
a) determining sensor data indicative of signals from each of the one or more sensors;
and,
b) causing the sensor data to be wirelessly transmitted to a monitoring device.

37) The system according to claim 36, wherein the sensor data is stored in a remote data store.

38) The system according to claim 36, wherein the at least one electronic processing device is configured to control at least one of the PAP device and one or more valves in the flow path in accordance with the determined sensor data to thereby regulate pressure and/or flow provided to the user.

39) The system according to claim 1, wherein the connector system includes:
a) an interface that is coupled to an extra-oral portion of the appliance that extends beyond the lips of the user in which the at least one extra-oral opening is provided;
b) an adaptor which is coupled to the interface;
c) a nasal tube holder which is coupled to the adaptor; and,
d) a nasal tube for receiving air from the PAP device that is removably attached to the nasal tube holder and which allows PAP to be delivered to the nose of the user.

40) The system according to claim 39, wherein the nasal tube holder includes one or more breathing ports in a front surface thereof in fluid communication with the at least one extra-oral opening of the appliance.

41) The system according to claim 40, wherein a one-way valve and/or heat and moisture exchanger is positioned behind one of the breathing ports of the nasal tube holder.

42) Apparatus for providing breathing assistance, the apparatus including a body for positioning within an oral cavity of a user, the body defining:
a) at least one first and second extra-oral opening configured to allow airflow between lips of the user;
b) at least two intra-oral openings provided in the oral cavity to allow air flow into and out of a posterior region of the oral cavity; and,
c) at least two channels, each channel connecting a respective intra-oral opening to at least one of the first and second extra-oral openings and each channel passing at least one of at least partially along the buccal cavity and at least partially between the teeth to thereby provide an airway for the user, the airway at least partially bypassing the nasal passage and acting to replicate a healthy nasal passage and pharyngeal space.

43) Apparatus according to claim 42, wherein a first airway is provided between a first extra-oral opening and a first intra-oral opening and a second airway is provided between a second extra-oral opening and a second intra-oral opening, and wherein the first airway is partitioned from the second airway.

44) Apparatus according to claim 43, wherein the first airway passes at least partially along the buccal cavity and the second airway passes at least one of:
a) at least partially between the user's maxillary and mandibular teeth; and,
b) at least partially between the user's maxillary and mandibular teeth and at least partially along the buccal cavity.

45) Apparatus according to claim 44, wherein the first airway and second airway are partitioned so that the first airway is positioned at least one of:
a) above the second airway; and,
b) to the outer lateral side of the second airway.

46) Apparatus according to any one of claims 43 to 45, wherein the first airway is used during inhalation and the second airway is used during exhalation.

47) Apparatus according to any one of claims 42 to 46, wherein the at least one first extra-oral opening is connectable to a supply of air from a positive airway pressure (PAP) device.

48) Apparatus according to claim 47, wherein the at least one first extra-oral opening is connectable to a connector system including a connector having a body in fluid communication with an inlet for receiving air from the PAP device and at least one outlet connected to the at least one first extra-oral opening for supplying the air thereto.

49) Apparatus according to claim 48, wherein the at least one second extra-oral opening is connectable to at least one opening in the connector body thereby allowing natural breathing through at least part of the connector.

50) Apparatus according to any one of claims 42 to 49, wherein the at least one second extra-oral opening is connectable to a sensor housing having at least one sensor for measuring an indication of at least one of:
a) body or head position;
b) air pressure;
c) air flow rate;
d) temperature;
e) moisture; and,
f) motion.

51) Apparatus according to any one of claims 42 to 50, wherein the at least one first and second extra-oral openings are provided in an extra-oral portion of the apparatus that extends beyond the lips of the user and has a generally elliptic cross section.

52) A connector system for connection to an oral appliance for providing breathing assistance to a user, the connector system including a connector having a body connectable to at least one extra-oral opening of the oral appliance, the body defining at least one passageway between an inlet for receiving air from a positive airway pressure (PAP) device and at least one outlet in fluid communication with the inlet for directing the air from the PAP device into at least one of the at least one extra-oral opening of the oral appliance and the nose of the user.

53) The connector system according to claim 52, wherein at least one inlet valve is provided downstream of the inlet for regulating pressure and/or flow through the connector.

54) The connector system according to claim 53, wherein a first inlet valve is provided to regulate pressure and/or flow into the appliance and a second inlet valve is provided downstream of the first inlet valve for regulating pressure and/or flow into a nasal connecting portion that directs air into the nose of the user.

55) The connector system according to any one of claims 52 to 54, wherein the connector body includes one or more first outlets that connect to one or more corresponding first extra-oral openings of the appliance to allow air from the PAP device into the appliance.

56) The connector system according to claim 55, wherein the connector body includes one or more openings that connect to one or more corresponding second extra-oral openings of the appliance to facilitate natural breathing.

57) The connector system according to claim 56, wherein the connector body includes one or more breathing ports in fluid communication with the one or more openings.

58) The connector system according to any one of claims 54 to 55, wherein the nasal connecting portion is connected to one or more second outlets of the connector body.

59) The connector system according to claim 58, wherein nasal prongs are connected to the nasal connection portion.

60) The connector system according to claim 59, wherein the nasal connecting portion and/or nasal prongs are at least one of rotatably and/or slidably movable relative to the connector body.

61) The connector system according to claim 59 or claim 60, wherein nasal pillows are inserted into the nasal prongs.

62) The connector system according to claim 61, wherein the nasal pillows are slidable movable with respect to the nasal prongs.

63) The connector system according to any one of claims 59 to 62, wherein exhalation ports are provided in the nasal connecting portion and/or nasal prongs.

64) The connector system according to any one of claims 52 to 63, further including one or more sensors positioned to monitor at least one of pressure and/or air flow to or from the appliance and/or nose.

65) The connector system according to any one of claims 52 to 64, wherein the connector body is 3D printed or moulded.

66) The connector system according to claim 52, wherein the connector has a multi-part body including:
a) an interface that is coupled to an extra-oral portion of the appliance that extends beyond the lips of the user in which the at least one extra-oral opening is provided;
b) an adaptor which is coupled to the interface;
c) a nasal tube holder which is coupled to the adaptor; and,
d) a nasal tube for receiving air from the PAP device that is removably attached to the nasal tube holder and which allows PAP to be delivered to the nose of the user.

67) A system for providing breathing assistance to a user, the system including:
a) an oral appliance including a body for positioning within an oral cavity of the user; and,
b) at least one extra-oral connector coupled to the oral appliance that at least one of:
i) modifies an airway;
ii) provides an airway; and,
iii) monitors an airway.

68) The system according to claim 67, wherein the at least one extra-oral connector houses at least one sensor for measuring an indication of at least one of:
a) body or head position;
b) air pressure;
c) air flow rate;
d) temperature;
e) moisture; and,
f) motion.

69) The system according to any one of claim 67 or claim 68, wherein the at least one extra-oral connector includes at least one of:
a) a valve/restrictor to provide resistance during exhalation; and,
b) a heat and moisture exchanger.

70) The system according to any one of claims 67 to 69, wherein the at least one sensor and/or valve is positioned proximate the user's mouth and/or nose.

71) The system according to any one of claims 67 to 70, wherein the body of the oral appliance defines at least one extra-oral opening for allowing airflow between lips of the user in fluid communication with at least one intra-oral opening provided in the oral cavity to direct airflow into and/or out of a posterior region of the oral cavity.

72) The system according to claim 71, wherein the body further defines at least two channels, each channel connecting an intra-oral opening to the at least one extra-oral opening, each channel passing at least one of at least partially along the buccal cavity and at least partially between the teeth to thereby provide an airway for the user, the airway at least partially bypassing the nasal passage and acting to replicate a healthy nasal passage and pharyngeal space.

73) The system according to any one of claims 67 to 72, wherein the at least one extra-oral connector includes a nasal connecting portion.

74) The system according to claim 73, wherein a pair of nasal pillows are coupled to the nasal connecting portion for insertion into a user's nostrils.

75) The system according to claim 74, wherein the nasal pillows are custom fit to the user as a result of one of:
a) heat setting a thermoplastic material;
b) bending a flexible tubing.

76) The system according to claim 75, wherein the flexible tubing is reinforced by ductile metal strips or coil.

77) The system according to any one of claims 72 to 76, wherein the at least one extra-oral connector is connected to the at least one extra-oral opening of the appliance and configured to receive air and/or oxygen from a positive airway pressure (PAP) device.

78) The system according to any one of claim 77, wherein air and/or oxygen from the PAP device is delivered to the user through at least one of:
a) the nose of the user;
b) the oral appliance via the airway; and,
c) the nose and the oral appliance via the airway.

79) The system according to claim 78, wherein the at least one extra-oral connector includes at least one control valve for directing air and/or oxygen to at least one of the mouth and/or nose of the user.

80) The system according to claim 78 or claim 79, wherein the at least one extra-oral connector includes at least one vent for controlling natural breathing through the mouth and/or nose.

81) The system according to any one of claims 72 to 80, wherein the channel on each side of the oral appliance is partitioned so as to define a split airway.

82) The system according to claim 81, wherein a first portion of the split airway is used for delivering PAP and a second portion of the split airway is used for natural breathing.

83) The system according to claim 67, wherein the extra-oral connector includes:
a) an interface that is coupled to an extra-oral portion of the appliance that extends beyond the lips of the user in which the at least one extra-oral opening is provided;
b) an adaptor which is coupled to the interface;
c) a nasal tube holder which is coupled to the adaptor; and,
d) a nasal tube for receiving air from the PAP device that is removably attached to the nasal tube holder and which allows PAP to be delivered to the nose of the user.
